(19) United States
(12) Patent Application Publication
(10) Pub. No.: US 2006/0115460 A1
(43) Pub. Date: Jun. 1, 2006

(54) Compositions and methods comprising WNT proteins to promote repair of damaged tissue

(76) Inventor: Gail K. Naughton, San Diego, CA (US)

Correspondence Address: DECHERT LLP, P.O. BOX 10004, PALO ALTO, CA, 94303, US

(21) Appl. No.: 11/216,580

(22) Filed: Aug. 30, 2005

Related U.S. Application Data
(60) Provisional application No. 60/606,072, filed on Aug. 30, 2004.
(60) Provisional application No. 60/691,731, filed on Jun. 17, 2005.

(51) Int. Cl. A61K 48/00; C12N 5/08

(52) U.S. Cl. 424/93.21; 435/366

(57) ABSTRACT

Compositions and methods for treating damaged tissue are provided herein.

### CROSS-REFERENCES TO RELATED APPLICATIONS

This application claims benefit under 35 U.S.C. §119(e) to application Ser. No. 60/606,072, entitled “Methods for Promoting Repair and Regeneration of Ischemic Tissues,” filed Aug. 30, 2004, and to application Ser. No. 60/691,731, entitled “Methods for Promoting Repair and Regeneration of Ischemic Tissues,” filed Jun. 17, 2005, the disclosures of which are incorporated herein by reference in their entireties.

### BACKGROUND

Development and maintenance of organ systems such as the vasculature requires the activity and coordination of a multitude of signaling pathways. The Wnt signaling pathway has been shown to play important roles both in normal development and in the pathogenesis of a variety of diseases, including cancer. Recent studies suggest that formation and remodeling of the vasculature during development and in pathological states, such as cancer, are regulated by Wnt signaling. Wnt factors are cysteine-rich, secreted glycoproteins that are highly conserved among species ranging from hydra to humans. Reception and transduction of Wnt signals involves binding of Wnt proteins to members of two distinct families of cell surface receptors, members of the Frizzled (Fz) gene family and members of the LDL-receptor-related protein (LRP) family. Fz proteins contain an extracellular cysteine-rich domain, seven transmembrane domains and a short cytoplasmic tail. At least 19 Wnt homologs and 10 Fz homologs are expressed in mammals. For more background regarding Wnt proteins and their signaling pathways, see, e.g., Goodwin and D-Amore, 2002, Angiogenesis, 5:1-9 and Miller J R, 2001, Genome Biology, 3:3001.1-3001.15.

### SUMMARY

The present disclosure provides methods and compositions for promoting repair and remodeling of damaged tissue. Examples of damaged tissue that can be treated using the methods described herein include, but are not limited to, intestinal tissue, cardiac tissue, liver tissue, kidney tissue, skeletal muscle, connective tissue, and/or skin tissue. The methods comprise administering to a subject an effective amount of the conditioned medium using methods know to the skilled artisan. For example, in some embodiments, compositions can be administered by injection, such as with a hypodermic needle.

The compositions comprise conditioned medium made from a three-dimensional tissue. The conditioned medium comprises a variety of bioactive agents, such as growth factors and/or Wnt proteins, secreted by the cells of three-dimensional tissue. The bioactive agents promote one or more biological processes that contribute to the effective repair of damaged tissue, including but not limited to, prevention and/or reduction in tissue thinning, as is characteristic of the tissue remodeling observed in ischemic tissue, and/or promotion of endothelialization, tissue growth, vascularization and/or angiogenesis. In some embodiments, the conditioned medium can be used to promote development and differentiation of hair follicles.

In some aspects, the compositions comprise isolated bioactive agents made by the three dimensional tissue(s). For example, in some embodiments, the compositions comprise isolated Wnt proteins produced by the three-dimensional tissue. One, some, or all of the repertoire of Wnt proteins elaborated by the three-dimensional tissue(s) can be used, including but not limited to, Wnt5a, Wnt7a, and Wnt 11. The Wnt proteins can be used independently, or in combination with other bioactive agents, such as growth factors, secreted by the three-dimensional tissue.

In other aspects, kits are provided comprising various pharmaceutical formulations comprising the conditioned medium. The conditioned medium can be provided in various dosage forms, such as injectable suspensions or lyophilizates for reconstitution with a suitable diluent for injection.

### BRIEF DESCRIPTION OF THE FIGURES

FIG. 1 shows the effect of three dimensional tissue conditioned medium on cell proliferation in four different epithelial cell types (Caco-2, NCI-H292, primary keratinocytes, and HT29). The conditioned medium enhanced cell growth in primary keratinocytes and NCI-H292 cells but not for the most part in Caco-2 or HT29 cells. Not all cell lines responded, and not all conditioned medium were effective.

FIG. 2 shows phase contrast microscopy of cells treated with conditioned media. Caco-2, NCI-H292 and epidermal keratinocyte (not shown) morphology is altered by the conditioned medium whereas HT-29 cells (not shown) were not affected. The apparent formation of dome like structure in the Caco-2 and NCI-H292 cell lines may indicate an enhancement or induction of differentiation, as mucin-production (a marker of both intestinal and respiratory epithelium) has been reported to lead to similar morphological changes in these cell lines as well as primary human intestinal and respiratory cells in culture.

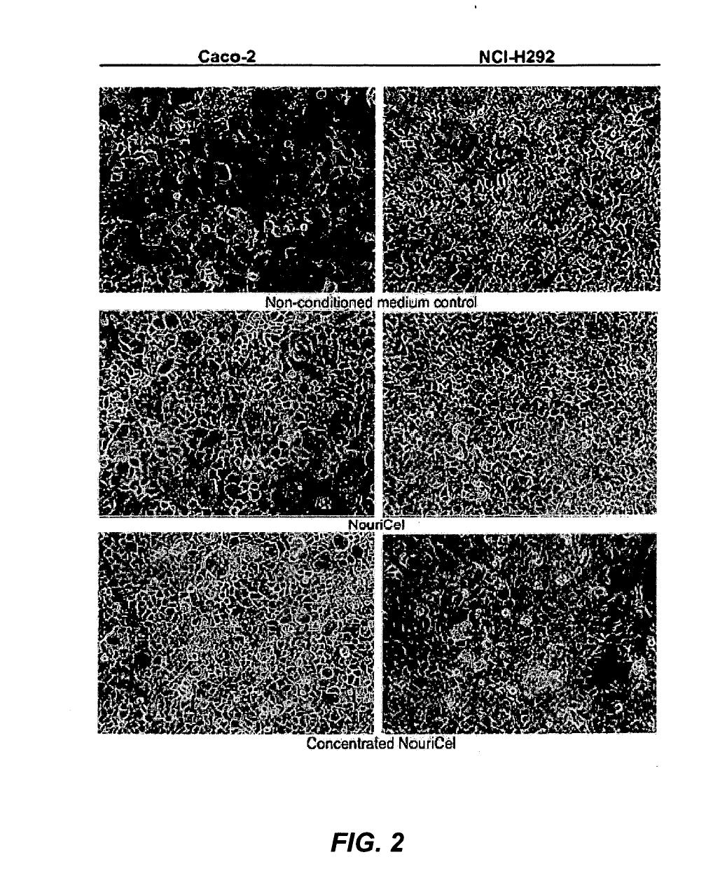

FIG. 3 shows analysis by immunofluorescence of adherens (ZO-1) and tight junction marker (claudin-1) in Caco-2 cells placed on collagen coated glass slides and treated with conditioned media from three dimensional stromal cell tissues. There is discontinuous staining for ZO-1 in the control medium panel (white arrow), and the junctional localization of claudin-1 in all the three dimensional tissue conditioned medium treated panels (dashed white arrow).

FIG. 4 shows the morphological changes in organotypic, high density microporous membrane cultures treated with conditioned medium as examined by transmission electron microscopy. A tissue section is shown for control and three-dimensional stromal tissue conditioned medium treated Caco-2 cells. There is increased overall thickness, more columnar shape, and an increase in intracellular spaces in the cells treated with the three dimensional tissue conditioned medium. These are all characteristics of normal differentiation of these cell types.

FIG. 5 shows a higher magnification transmission electron microscope analysis of effects of three dimensional stromal tissue conditioned medium <10 kD permeate on Caco-2 cells in high density organotypic cultures. There is an increase in cellular processes, microvilli, and mitochondrial location (apical in the three dimensional stromal tissue conditioned medium sample). Tight junctions were less frequent in the three dimensional tissue conditioned medium sample than the control.

FIG. 6 is a TEM analysis of Three-dimensional stromal tissue conditioned medium <10 kD permeate on Caco-2 cells in high density organotypic cultures showing effects on cellular processes, apical microvilli, and dense glycogen deposits

FIG. 7 shows the effect of injecting conditioned medium on stimulation of hair growth in adult C57B1/6 mice. Hair growth was examined 30 days after injection.

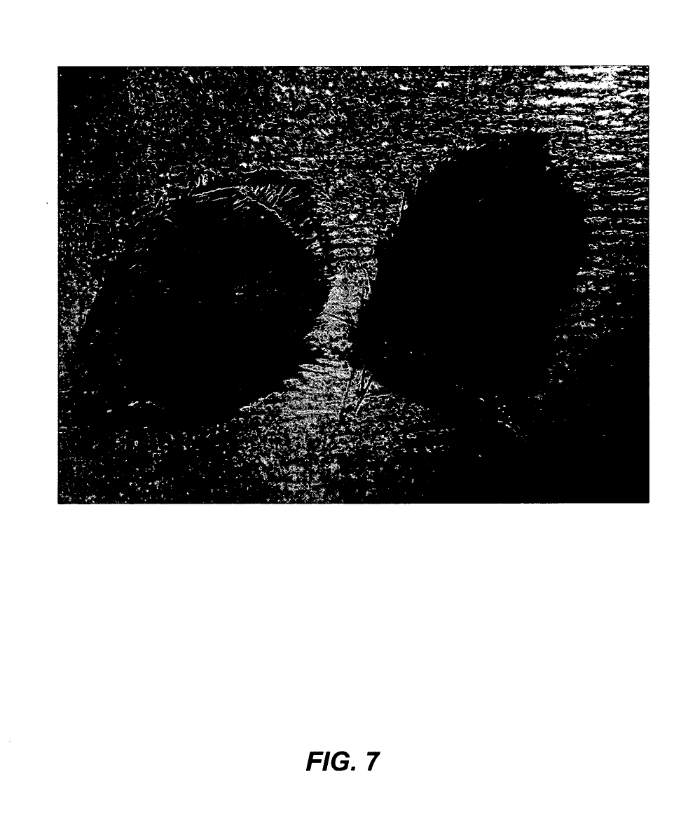

FIG. 8 shows C57B1/6 mice treated with conditioned medium.

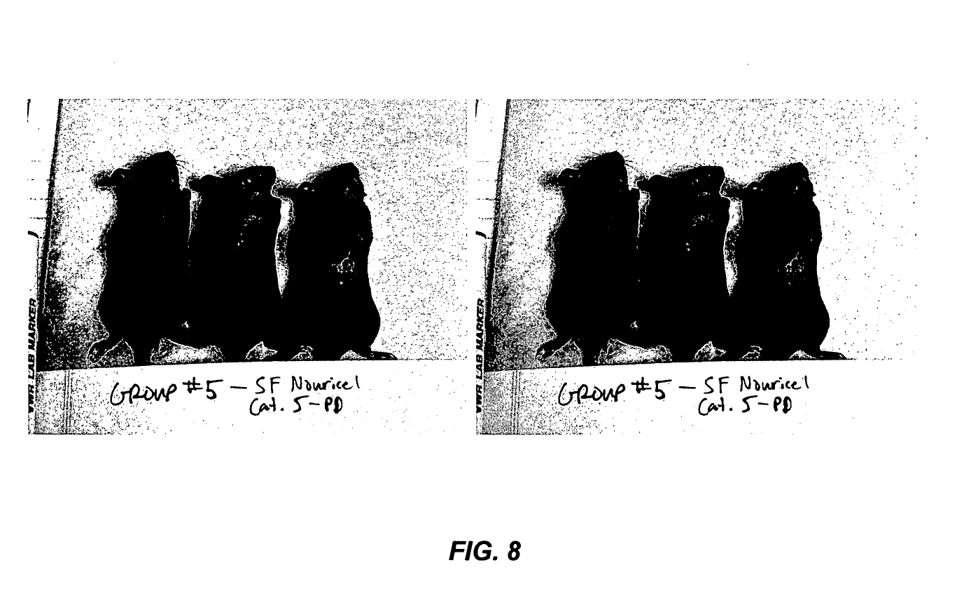

FIG. 9 shows skin pigmentation changes after 30 days from injected conditioned medium.

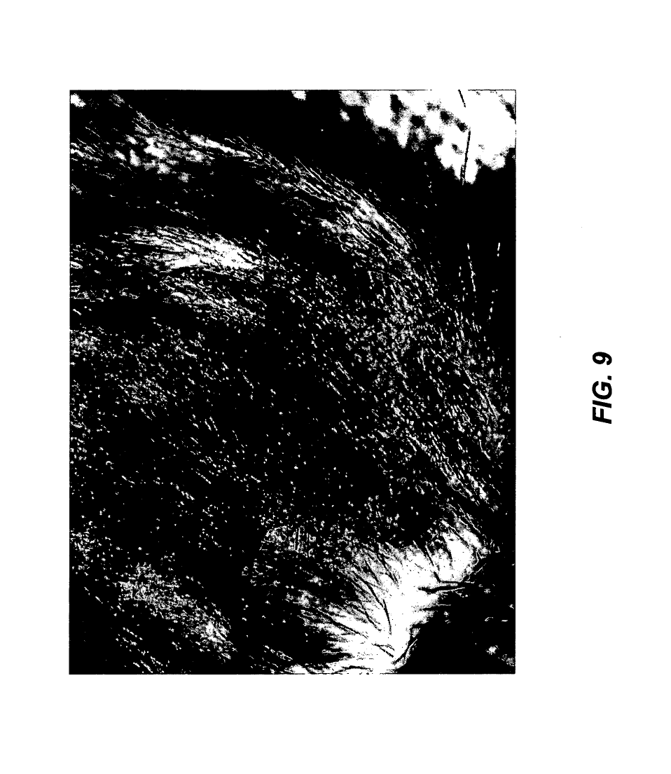

FIG. 10 shows histological examination of the skin in animals treated with conditioned medium.

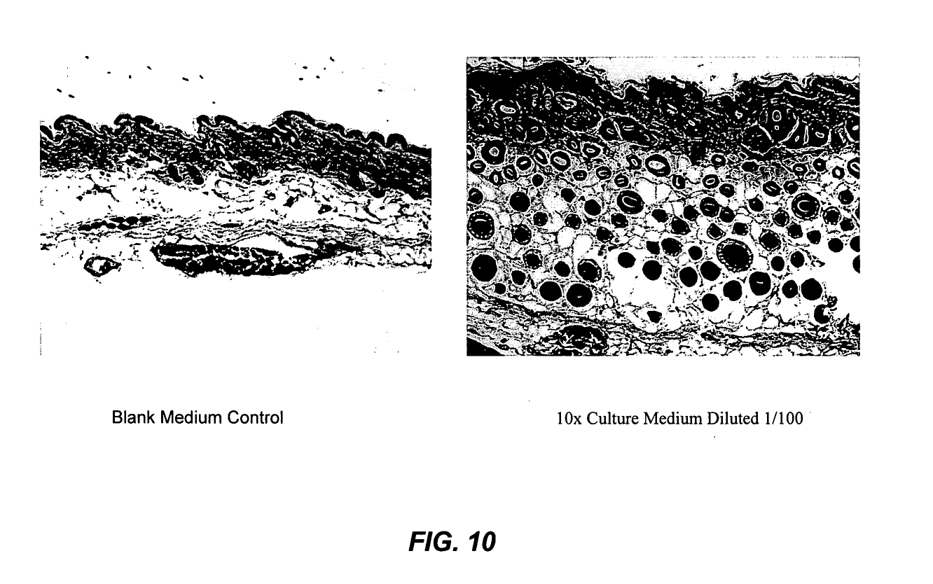

FIG. 11 is a histological comparison (using trichome stain) after 14 days showing that injection of conditioned medium induced hair follicles after 14 days in mice receiving 10× conditioned medium diluted 1/100 as compared to blank medium controls.

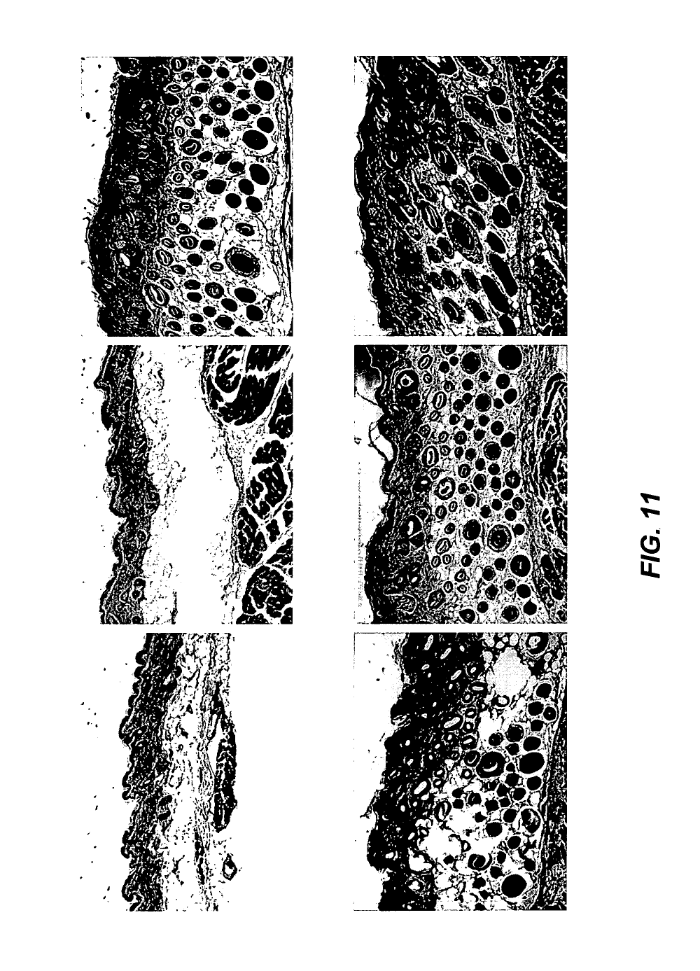

FIG. 12 shows that hair follicle anagen induction by the conditioned medium is confined to the site of injection.

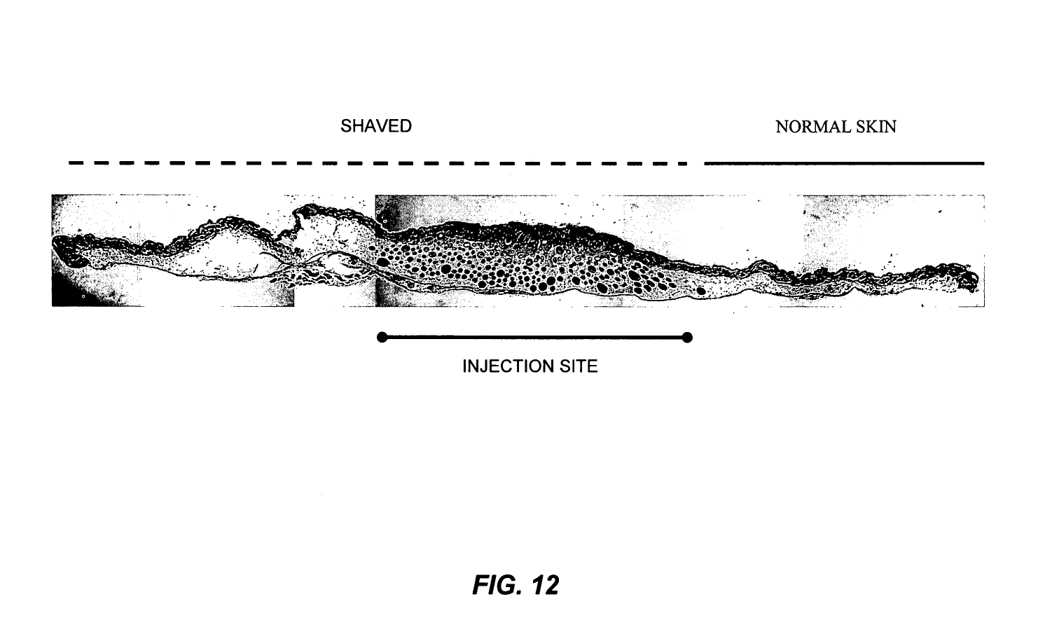

FIG. 13 shows cross sections of follicles induced by injection of conditioned medium. The follicles exhibit normal anagen morphology, including connective tissue sheath, outer root sheath, inner root sheath, cortex, matrix, and dermal papilla.

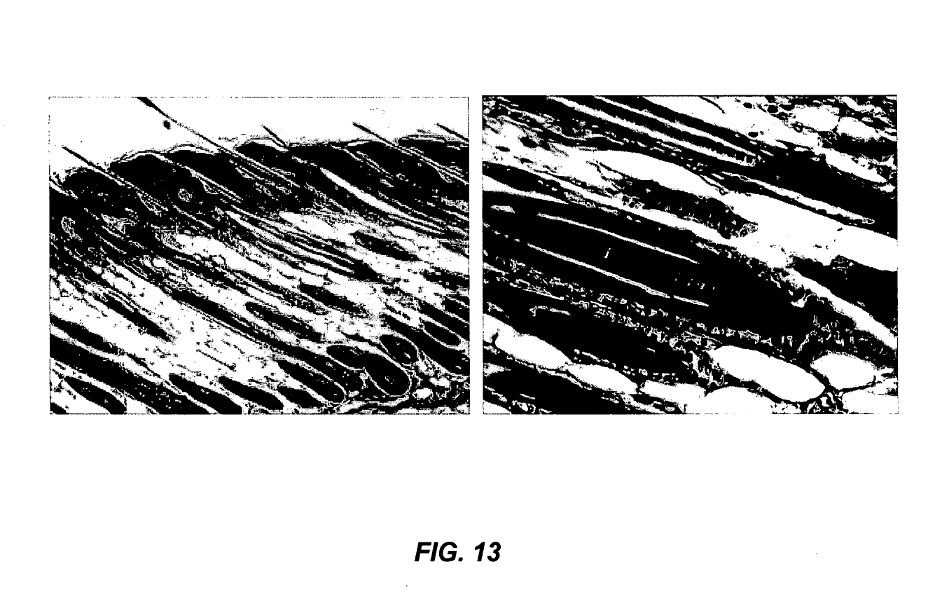

FIG. 14 shows Keratin 15 and Keratin 10 expression in conditioned medium-induced hair follicles, evidencing of normal, mature hair follicles.

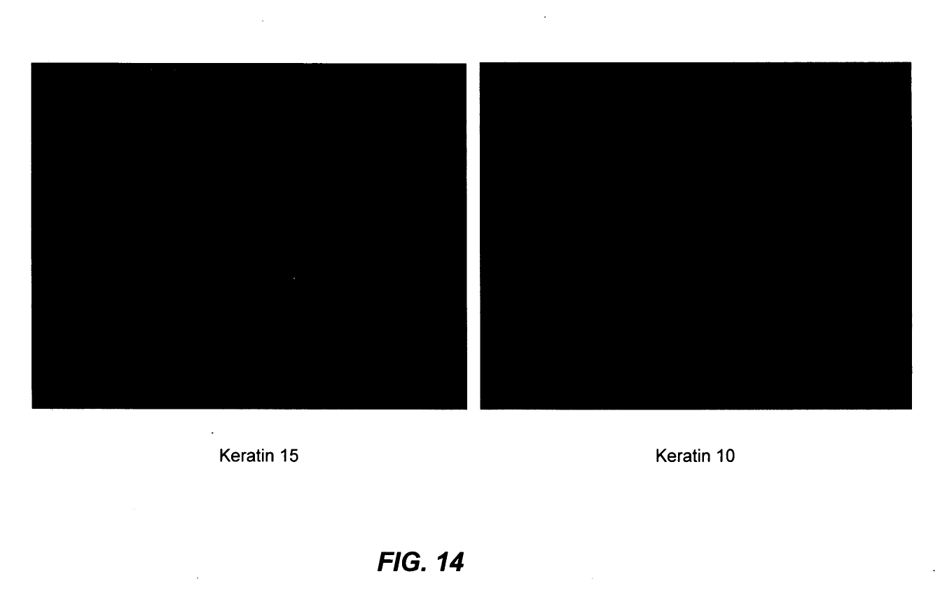

FIG. 15 shows Wnt signaling in epidermal keratinocyles in vitro. Nuclear translocation of β-catenin is induced by conditioned medium, providing evidence that the conditioned medium contains Wnt proteins.

FIG. 16 shows a histological evaluation of hair follicle formation in adult SCID mice. Left panels are tissue sections of animals injected with blank medium control and the right panels show tissue sections of animals injected with three dimensional tissue conditioned medium. Adult SCID mouse were injected once subcutaneously (SQ) and the animals examined after 10 days.

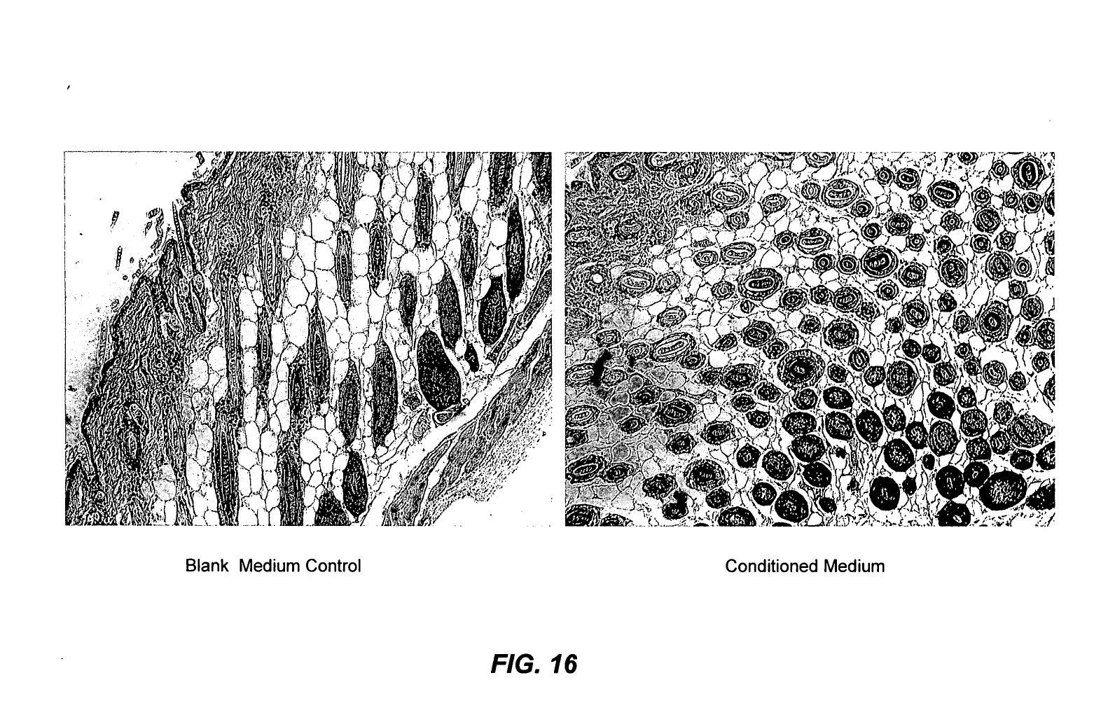

FIG. 17 shows quantification of follicular structures induced by three dimensional stromal tissue conditioned medium after 14 days. The y-axis is number of follicle-like structures in the histology field.

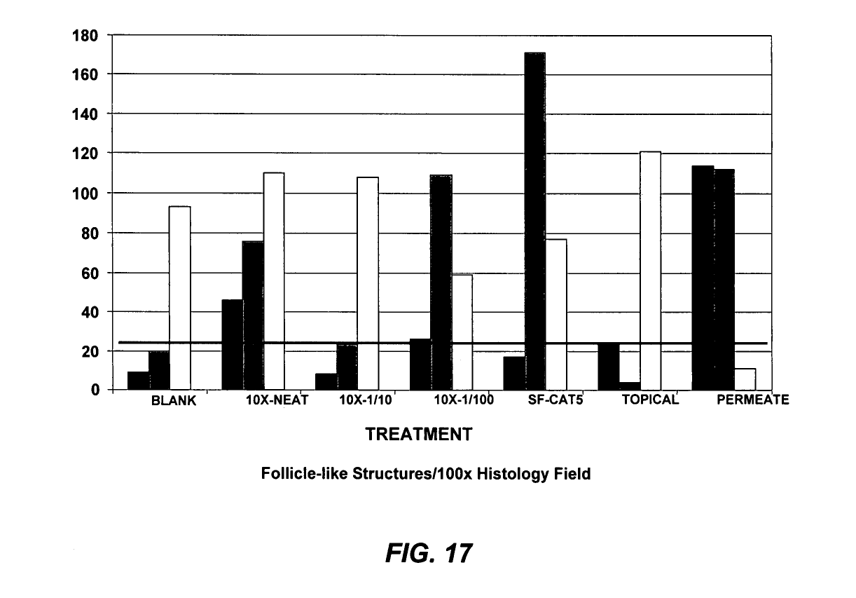

FIG. 18 shows combined data quantifying the follicular structures (N=6).

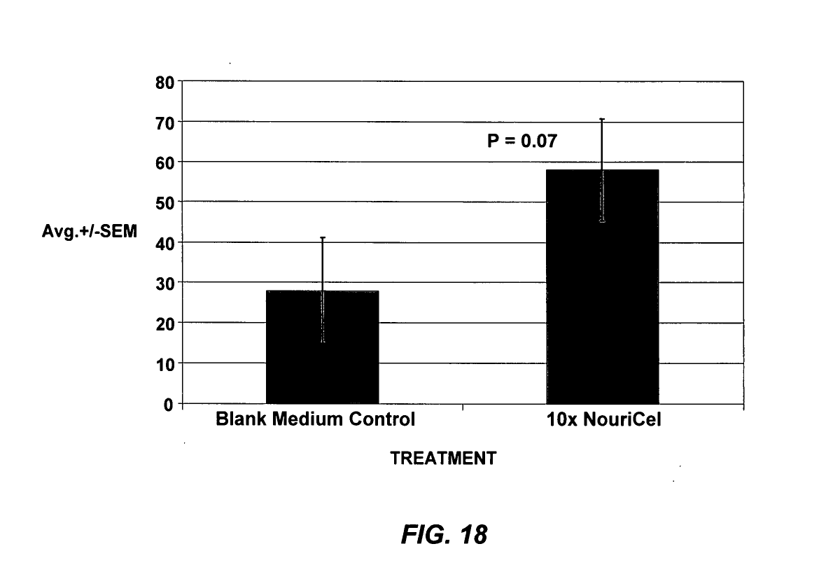

### DETAILED DESCRIPTION

The compositions and methods described herein have a variety of applications including, but not limited to, promoting repair and remodeling of damaged tissue, including intestinal tissue, cardiac tissue, liver tissue, kidney tissue, skeletal muscle, connective tissue, skin tissue, and/or other tissues. Generally, the methods described herein comprise administering to a subject an effective amount of a composition comprising a conditioned medium made from a three-dimensional tissue. Without being limited to any theory, the conditioned medium is believed to promote tissue repair when provided to a damaged tissue due to the presence of bioactive agents, such as Wnt proteins and/or other growth factors.

Three Dimensional Tissue and Scaffolds

In various embodiments, the conditioned medium capable of promoting the repair of damaged tissue is obtained from a three dimensional tissue. Generally, the cells are cultured on a scaffold, also referred to herein as a scaffold, composed of a biocompatible, non-living material. The scaffold can be of any material and/or shape that: (a) allows cells to attach to it (or can be modified to allow cells to attach to it); and (b) allows cells to grow in more than one layer (i.e., form a three dimensional tissue).

In some embodiments, the biocompatible material is formed into a three-dimensional scaffold, comprising interstitial spaces for attachment and growth of cells into a three dimensional tissue. The openings and/or interstitial spaces of the scaffold are an appropriate size to allow the cells to stretch across the openings or spaces. Maintaining actively growing cells stretched across the scaffold appears to enhance production of the repertoire of bioactive agents responsible for the activities described herein. If the openings are too small, the cells can rapidly achieve confluence but be unable to easily exit from the mesh. These trapped cells can exhibit contact inhibition and cease production of the repertoire of bioactive agents described herein, as well as other factors useful in supporting the proliferation and maintenance of the cultures. If the openings are too large, the cells may be unable to stretch across the opening and production of the repertoire of bioactive agents described herein, as well as other factors useful in supporting the proliferation and maintenance of the cultures may be decreased. When using a mesh type of scaffold, as exemplified herein, it has been found that openings at least about 140 μm, at least about 150 μm, at least about 160 μm, at least about 175 μm, at least about 185 μm, at least about 200 μm, at least about 210 μm, and at least about 220 μm work satisfactorily. However, depending upon the intricacy of the scaffold, other sizes can work equally well. In fact, any shape or structure that allows the cells to stretch, continue to replicate, and grow for a suitable length of time can function to elaborate the bioactive agents in accordance with the methods described herein.

In some embodiments, the three dimensional scaffold is formed from material of polymers or threads that are braided, woven, knitted or otherwise arranged to form a scaffold, such as a mesh or fabric. The materials can be formed by casting of the material or fabrication into a foam, matrix, or sponge-like scaffold. In other embodiments, the three dimensional scaffold is in the form of matted fibers made by pressing polymers or other fibers together to generate a material with interstitial spaces. The three dimensional scaffold can take any form or geometry for the growth of cells in culture as long as the conditioned medium produced there from displays at least one of the tissue repair promoting activities described herein. Descriptions for cell cultures using a three dimensional scaffold are described in U.S. Pat. Nos. 6,372,494; 6,291,240; 6121,042; 6,022,743; 5,962,325; 5,858,721; 5,830,708; 5,785,964; 5,624,840; 5,512,475; 5,510,254; 5,478,739; 5,443,950; and 5,266,480, the disclosures of which are incorporated herein by reference in their entireties. As further described below, other types of scaffolds can suffice for generating the appropriate conditioned medium.

A number of different materials can be used to form the scaffold. These materials include non-polymeric and polymeric materials. Polymeric materials can be any type of block polymers, co-block polymers (e.g., di, tri, etc.), linear or branched polymers, crosslinked or non-crosslinked. Non-limiting examples of materials for use as scaffolds include, among others, glass fiber, polyethylene, polypropylene, polyamides (e.g., nylon), polyesters (e.g., Dacron), polystyrenes, polyacrylates, polyvinyl compounds (e.g., polyvinylchloride; PVC), polycarbonates, polytetrafluorethylenes (PTFE; TEFLON), expanded PTFE (ePTFE), thermanox (TPX), nitrocellulose, polysaacharides (e.g., celluloses, chitosan, agarose), polypeptides (e.g., silk, gelatin, collagen), polyglycolic acid (PGA), and dextran.

In some embodiments, the scaffold comprises materials that degrade over time under the conditions of use. As used herein, a degradable material refers to a material that degrades or decomposes. In some embodiments, the degradable material is biodegradable, i.e., degrades through the action of biological agents, either directly or indirectly. Non-limiting examples of biodegradable materials include, among others, poly(lactic-co-glycolic acid) (i.e., PLGA), polyethylene terephtalate (PET), trimethylene carbonate (TMC), co-polymers of TMC, PGA, and/or PLA, polycaprolactone, catgut suture material, collagen (e.g., equine collagen foam), polylactic acid, or hyaluronic acid. For example, these materials can be woven into a three-dimensional scaffold such as a collagen sponge or collagen gel.

In embodiments in which the cultures are to be maintained for long periods of time, cryopreserved, and/or where additional structural integrity is desired, the three dimensional scaffold typically comprises nondegradable materials. As used herein, nondegradable materials refer to materials that do not degrade or decompose significantly under the conditions used to culture the three-dimensional tissue. Exemplary nondegradable materials, include, but are not limited to, nylon, dacron, polystyrene, polyacrylates, polyvinyls, teflons, and cellulose. An exemplary nondegrading three dimensional scaffold comprises a nylon mesh, available under the tradename Nitex®, a nylon filtration mesh having an average pore size of 140 μm and an average nylon fiber diameter of 90 μm (#3-210/36, Tetko, Inc., N.Y.).

In other embodiments, the three dimensional scaffold is a combination of degradeable and non-degradeable materials. The non-degradable material provides stability to the scaffold during culturing, while the degradeable material allows interstitial spaces to form sufficient for formation of three-dimensional tissue that produces bioactive agents sufficient for promoting the repair of damaged tissue. The degradable material can be coated onto the non-degradable material or woven, braided or formed into a mesh. Various combinations of degradable and non-degradable materials can be used. An exemplary combination is poly(ethylene therephtalate) (PET) fabrics coated with a thin biodegradable polymer film (poly[D-L-lactic-co-glycolic acid] PLGA), in order to obtain a polar structure.

In various embodiments, the scaffold material can be pre-treated prior to inoculation with cells to enhance cell attachment to the scaffold. For example, prior to inoculation with cells, nylon screens can be treated with 0.1 M acetic acid, and incubated in polylysine, fetal bovine serum, and/or collagen to coat the nylon. In some embodiments, polystyrene can be analogously treated using sulfuric acid. In other embodiments, the growth of cells in the presence of the three-dimensional scaffold is further enhanced by adding to the scaffold, or coating with proteins (e.g., collagens, elastin fibers, reticular fibers), glycoproteins, glycosaminoglycans (e.g., heparan sulfate, chondroitin-4-sulfate, chondroitin-6-sulfate, dermatan sulfate, keratan sulfate, etc.), fibronectins, a cellular matrix, and/or other materials glycopolymer (poly[N-p-vinylbenzyl-D-lactoamide], PVLA) in order to improve cell attachment. Treatment of the scaffold or scaffold is useful to improve attachment of cells.

In other embodiments, the scaffold for generating the three dimensional tissue comprises particles so dimensioned that tissues cultured in presence of the particles elaborate the bioactive agents that promote tissue repair. In some embodiments, the particles comprise microparticles, or other suitable particles, such as microcapsules and nanoparticles, which can be biodegradable or non-biodegradable (see, e.g., “Microencapsulates: Methods and Industrial Applications,” in Drugs and Pharmaceutical Sciences, 1996, Vol 73, Benita, S. ed, Marcel Dekker Inc., New York, and U.S. application Ser. No. ______ entitled “Cultured Three Dimensional Tissues and Uses Thereof,” filed concurrently herewith, the disclosure of which is incorporated herein by reference in its entirety). Generally, the microparticles have a particle size range of at least about 1 μm, at least about 10 μm, at least about 25 μm, at least about 50 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1000 μm. Nanoparticles have a particle size range of at least about 10 nm, at least about 25 nm, at least about 50 nm, at least about 100 nm, at least about 200 nm, at least about 300 nm, at least about 400 nm, at least about 500 nm, at least about 600 nm, at least about 700 nm, at least about 800 nm, at least about 900 nm, at least about 1000 nm. The particles can be porous or nonpororus. Various particle formulations can be used for preparing the three dimensional tissues, including particles made from biodegradable or non-biodegradable materials used to form the mesh or woven polymers described above. See, also, U.S. application Ser. No. ______ entitled “Cultured Three Dimensional Tissues and Uses Thereof,” filed concurrently herewith, the disclosure of which is incorporated herein by reference in its entirety.

Exemplary non-biodegradable particles include, but are not limited to, polysulfones, poly (acrylonitrile-co-vinyl chloride), ethylene-vinyl acetate, hydroxyethylmethacrylate-methyl-methacrylate copolymers. Biodegradable particles include those made from fibrin, casein, serum albumin, collagen, gelatin, lecithin, chitosan, alginate or poly-amino acids such as poly-lysine. Biodegradable synthetic polymers include polylactide (PLA), polyglycolide (PGA), poly (lactide-co-glycolide) (PLGA), poly (caprolactone), polydioxanone trimethylene carbonate, polyhybroxyalkonates (e. g., poly (y-hydroxybutyrate)), poly (Y-ethyl glutamate), poly (DTH iminocarbony (bisphenol A iminocarbonate), poly (ortho ester), and polycyanoacrylate.

Hydrogels are also useful for delivering the bioactive agents. Generally, hydrogels are crosslinked, hydrophilic polymer networks. Non-limiting examples of polymers useful in hydrogel compositions include, among others, those formed from polymers of poly (lactide-co-glycolide), poly (N-isopropylacrylamide); poly (methacrylic acid-y-polyethylene glycol); polyacrylic acid and poly (oxypropylene-co-oxyethylene) glycol; and natural compounds such as chrondroitan sulfate, chitosan, gelatin, fibrinogen, or mixtures of synthetic and natural polymers, for example chitosan-poly (ethylene oxide). The polymers are typically crosslinked reversibly or irreversibly to form gels sufficient for cells to attach and form a three dimensional tissue.

Various methods for making particles are well known in the art, including solvent removal process (see, e.g., U.S. Pat. No. 4,389,330); emulsification and evaporation (Maysinger et al., 1996, Exp. Neuro. 141: 47-56; Jeffrey et al., 1993, Pharm. Res. 10: 362-68), spray drying, and extrusion methods. Exemplary particles for preparing three dimensional tissues are also described in US Published Application No. 2003/0211083 and U.S. Pat. Nos. 5,271,961; 5,413,797; 5,650,173; 5,654,008; 5,656,297; 5,114,855; 6,425,918; and 6,482,231;and the U.S. application Ser. No. ______ entitled “Cultured Three Dimensional Tissues and Uses Thereof,” filed concurrently herewith, the disclosures of which are incorporated herein by reference in their entireties.

It is to be understood that materials in various geometric forms other than those described above, can be used to generate three dimensional tissues capable of producing conditioned medium with tissue repair promoting characteristics, and thus the materials are not limited to the embodiments disclosed herein.

Cells and Culture Conditions

To form the three dimensional tissue, the biocompatible materials forming the scaffold are inoculated with the appropriate cells and grown under conditions suitable to promote production of a conditioned medium with tissue repair promoting properties. Cells can be obtained directly from a donor, from cell cultures made from a donor, or from established cell culture lines. In some instances, cells can be obtained in quantity from any appropriate cadaver organ or fetal sources. In some embodiments, cells of the same species and preferably matched at one or more MHC loci are obtained by biopsy, either from the subject or a close relative, which are then grown to confluence in culture using standard conditions and used as needed. The characterization of the donor cells are made in reference to the subject being administered, the conditioned medium and/or its isolated components.

Accordingly, in some embodiments, the cells are autologous, which refers to cells that originate from the intended recipient. Because the products in the conditioned media derive from the recipient's own cells, the possibility of an immunological reaction that neutralizes the activity of the conditioned media can be reduced. In these embodiments, cells are typically cultured to obtain a sufficient number to produce the conditioned media.

In other embodiments, the cells are obtained from a donor who is not the intended recipient of the culture medium. In some of these embodiments, the cells are syngeneic, derived from a donor who is genetically identical at all MHC loci. In other embodiments, the cells are allogeneic, derived from a donor differing at at least one MNC locus from the intended recipient. When the cells are allogeneic, the cells can be from a single donor or comprise a mixture of cells from different donors who themselves are allogeneic to each other. In further embodiments, the cells comprise xenogenic, i.e., the are derived from a species that is different from the intended recipient.

In various embodiments herein, the cells inoculated onto the scaffold are stromal cells typically comprising fibroblasts. In typical embodiments, stromal cells are derived from connective tissue, including, but not limited to: (1) bone; (2) loose connective tissue, including collagen and elastin; (3) the fibrous connective tissue that forms ligaments and tendons, (4) cartilage; (5) the extracellular matrix of blood; and (6) adipose tissue, which comprises adipocytes.

In various embodiments, stromal cells can be derived from various tissues or organs, such as skin, heart, blood vessels, skeletal muscle, liver, pancreas, brain, foreskin, which can be obtained by biopsy (where appropriate) or upon autopsy.

The fibroblasts can be from a fetal, neonatal, adult origin, or a combination thereof. In some embodiments, the stromal cells comprise fetal fibroblasts, which can support the growth of a variety of different cells and/or tissues. As used herein a fetal fibroblast refers to fibroblasts derived from fetal sources. As used herein neonatal fibroblast refers to fibroblasts derived from newborn sources. Under appropriate conditions, fibroblasts can give rise to other cells, such as bone cells, fat cells, and smooth muscle cells and other cells of mesodermal origin. In some embodiments, the fibroblasts comprise dermal fibroblasts. As used herein, dermal fibroblasts refers to fibroblasts derived from skin. Normal human dermal fibroblasts can be isolated from neonatal foreskin. These cells are typically cryopreserved at the end of the primary culture.

In some embodiments, fibroblasts are inoculated onto the scaffold. The fibroblasts can be from a fetal, neonatal, adult origin, or a combination thereof.

In other embodiments, the three-dimensional tissue can be made using stem or progenitor cells, either alone, or in combination with any of the cell types discussed herein. The term “stem cell” includes, but is not limited to, embryonic stem cells, hematopoietic stem cells, neuronal stem cells, and mesenchymal stem cells.

In some embodiments, a “specific” three-dimensional tissue can be prepared by inoculating the three-dimensional scaffold with cells derived from a particular organ, i.e., skin, heart, and/or from a particular individual who is later to receive the cells and/or tissues grown in accordance with the methods described herein.

In certain embodiments, in addition to stromal cells, other cell types can be present in the three dimensional tissue. Additional cell types can include, but are not limited to, smooth muscle cells, cardiac muscle cells, endothelial cells or skeletal muscle cells. In addition to fibroblasts, other cells can be added to form the three dimensional tissue required to support long term growth in culture. For example, other cells found in loose connective tissue can be inoculated onto the three-dimensional scaffold along with, or instead of, fibroblasts. Suitable cells include, but are not limited to, endothelial cells, pericytes, macrophages, monocytes, adipocytes, skeletal muscle cells, smooth muscle cells, and/or cardiac muscle cells. The additional cell types can readily be derived from appropriate tissues or organs such as skin, heart, and blood vessels, using methods known in the art, including those discussed above.

In some embodiments, one or more of the additional cell types described above can be inoculated onto the three-dimensional scaffold in the absence of fibroblasts. In other embodiments, one or more of the additional cell types described above can be inoculated onto the three-dimensional scaffold in combination with fibroblast cells. The fibroblast cells can be fetal in origin, neonatal in origin, adult in origin, or a combination thereof.

Cells, such as stromal cells, can be readily isolated by disaggregating an appropriate organ or tissue. This can be readily accomplished using techniques known to those skilled in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. These include, but are not limited to, trypsin, chymotrypsin, collagenase, elastase, and/or hyaluronidase, DNase, pronase, and dispase. Mechanical disruption can be accomplished by a number of methods including, but not limited to, the use of grinders, blenders, sieves, homogenizers, pressure cells, or insonators, to name but a few. For a review of tissue disaggregation techniques, see Freshney, Culture of Animal Cells. A Manual of Basic Technique, 2d Ed., A.R. Liss, Inc., New York, 1987, Ch. 9, pp. 107-126.

Once the tissue has been reduced to a suspension of individual cells, the suspension can be fractionated into subpopulations from which, in various embodiments, the fibroblasts and/or other stromal cells can be obtained. This can be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis and fluorescence-activated cell sorting. For a review of clonal selection and cell separation techniques, see Freshney, Culture of Animal Cells. A Manual of Basic Techniques, 2d Ed., A.R. Liss, Inc., New York, 1987, Ch. 11 and 12, pp. 137-168.

The isolation of cells, such as stromal cells can, for example, be carried out as follows: fresh tissue samples are thoroughly washed and minced in Hanks balanced salt solution (HBSS) in order to remove serum. The minced tissue is incubated from 1-12 hours in a freshly prepared solution of a dissociating enzyme such as trypsin. After incubation, the dissociated cells can be suspended, pelleted by centrifugation and plated onto culture dishes. As stromal cells attach before other cells, appropriate stromal cells can be selectively isolated and grown. The isolated stromal cells can be grown to confluency, lifted from the confluent culture and inoculated onto the three-dimensional scaffold (U.S. Pat. No. 4,963,489; Naughton et al., 1987, J. Med. 18(3&4):219-250). Inoculation of the three-dimensional scaffold with a high concentration of cells, e.g., approximately 1×10⁶ to 5×10⁷ stromal cells/ml, can result in the establishment of a three-dimensional tissue in shorter periods of time.

After inoculation, the cells are incubated in an appropriate nutrient medium that supports growth of cells into the three dimensional tissue. Many commercially available media such as Dulbecco's Modified Eagles Medium (DMEM), RPMI 1640, Fisher's, Iscove's, and McCoy's, are suitable for use. The medium can be supplemented with additional salts, carbon sources, amino acids, serum and serum components, vitamins, minerals, reducing agents, buffering agents, lipids, nucleosides, antibiotics, attachment factors, and growth factors. Formulations for different types of culture media are described in reference works available to the skilled artisan (e.g., Methods for Preparation of Media, Supplements and Substrates for Serum Free Animal Cell Cultures, Alan R. Liss, New York (1984); Tissue Culture: Laboratory Procedures, John Wiley & Sons, Chichester, England (1996); Culture of Animal Cells, A Manual of Basic Techniques, 4^{th} Ed., Wiley-Liss (2000). Typically, the three-dimensional tissue is suspended in the medium during the incubation period in order to enhance proliferative activity and secretion of bioactive agents, such as growth factors (soluble and matrix bound), cytokines, and Wnt proteins. In some embodiments, the culture can be “fed” periodically to remove spent media, depopulate released cells, and add fresh medium. During the incubation period, the cultured cells grow linearly along and envelop the filaments of the three-dimensional scaffold before beginning to grow into the openings of the scaffold.

Different proportions of the various types of collagen deposited on the scaffold can affect the growth of the cells that come in contact with the three dimensional scaffold. The proportions of extracellular matrix (ECM) proteins deposited can be manipulated or enhanced by selecting fibroblasts which elaborate the appropriate collagen type. This can be accomplished using monoclonal antibodies of an appropriate isotype or subclass that is capable of activating complement, and which define particular collagen types. These antibodies and complement can be used to negatively select the fibroblasts which express the desired collagen type. Alternatively, the cells used to inoculate the scaffold can be a mixture of cell types, such as a mixture of stromal cell types, which synthesize the appropriate collagen types desired. The distribution and origins of the various types of collagen is shown in Table I.

TABLE I
Distribution and Origin of Various Types of Collagen

| Collagen | | |
|---|---|---|
| Type | Principle Tissue Distribution | Cells of Origin |
| I | Loose and dense ordinary | Fibroblasts and reticular |
| | connective tissue; collagen | cells; smooth muscle cells |
| | fibers | |
| | Fibrocartilage | |
| | Bone | Osteoblast |
| | Dentin | Odontoblasts |
| II | Hyaline and elastic cartilage | Chondrocytes |
| | Vitreous body of the eye | Retinal cells |
| III | Loose connective tissue; | Fibroblasts and reticular cells |
| | reticular fibers | |
| | Papillary layer of dermis | |
| | Blood vessels | Smooth muscle cells; |
| | | endothelial cells |
| IV | Basement membranes | Epithelial and endothelial |
| | | cells |
| | Lens capsule of the eye | Lens fiber |
| V | Fetal membranes; placenta | Fibroblasts |
| | Basement membranes | |
| | Bone | |
| | Smooth muscle | Smooth muscle cells |
| VI | Connective tissue | Fibroblasts |
| VII | Epithelial basement | Fibroblasts; keratinocytes |
| | membranes; anchoring fibrils | |
| VIII | Cornea | Corneal fibroblasts |
| IX | Cartilage | |
| X | Hypertrophic cartilage | |
| XI | Cartilage | |
| XII | Papillary dermis | Fibroblasts |
| XIV | Reticular dermis | Fibroblasts |
| (undulin) | | |
| XVII | P170 bullous pemphigoid | Keratinocytes |
| | antigen | |

In various embodiments, the three dimensional tissues are characterized by a repertoire of bioactive agents that are produced by the cells. These bioactive agents can include growth factors (soluble and matrix bound), cytokines, and/or Wnt proteins. A description of the various bioactive agents that can be produced by the cultured three-dimensional tissues is provided below in the section entitled “Bioactive Agents Produced From Cultured Three Dimensional Tissues.”

The expression and/or secretion of various bioactive agents, including growth factors soluble and matrix bound), cytokines, and/or Wnt proteins, by the three dimensional tissue can be modulated by incorporating cells that release different levels of the factors of interest. For example, vascular smooth muscle cells, are known to produce substantially more VEGF than human dermal fibroblasts. By utilizing vascular smooth muscle cells instead of, or in addition to fibroblasts, for example, the expression and/or secretion of VEGF by the three dimensional tissue can be modulated.

Bioactive Agents Produced From Cultured Three Dimensional Tissues

In various embodiments, the three dimensional tissues are characterized by a repertoire of bioactive agents that are produced by the tissues. These bioactive agents can include growth factors (soluble and matrix bound), cytokines, and/or Wnt proteins.

In some embodiments, the three dimensional tissues are characterized by the expression and/or secretion of the growth factors listed in Table II.

TABLE II
Three Dimensional Cell Tissue Expressed Growth Factors and Cytokines

| | | Secreted Amount |
|---|---|---|
| Growth Factor | Expressed by Q-RT-PCR | Determined by ELISA |
| VEGF | 8 × 10⁶ copies/ug RNA | 700 pg/10⁶ cells/day |
| PDGF A chain | 6 × 10⁵ copies/ug RNA | |
| PDGF B chain | 0 | 0 |
| IGF-1 | 5 × 10⁵ copies/ug RNA | |
| EGF | 3 × 10³ copies/ug RNA | |
| HBEGF | 2 × 10⁴ copies/ug RNA | |
| KGF | 7 × 10⁴ copies/ug RNA | |
| TGF-f31 | 6 × 10⁶ copies/ug RNA | 300 pg/10⁶ cells/day |
| TGF-f33 | 1 × 10⁴ copies/ug RNA | |
| HGF | 2 × 10⁴ copies/ug RNA | 1 ng/10⁶ cells/day |
| IL-1a | 1 × 10⁴ copies/ug RNA | Below detection |
| IL-1b | 0 | |
| TNF-a | 1 × 10⁷ copies/ug RNA | |
| TNFF-b | 0 | |
| IL-6 | 7 × 10⁶ copies/ug RNA | 500 pg/10⁶ cells/day |
| IL-8 | 1 × 10⁷ copies/ug RNA | 25 ng/10⁶ cells/day |
| IL-12 | 0 | |
| IL-15 | 0 | |
| NGF | 0 | |
| G-CSF | 1 × 10⁴ copies/ug RNA | 300 g/10⁶ cells/day |
| Angiopoietin | 1 × 10⁴ copies/ug RNA | |

In some embodiments, the three-dimensional tissues are characterized by the expression and/or secretion of connective tissue growth factor (CTGF). See, e.g., Luo, Q., et al., 2004, J. Biol. Chem., 279:55958-68; Leask and Abraham, 2003, Biochem Cell Biol, 81:355-63; Mecurio, S. B., et al., 2004, Development, 131:2137-47; and, Takigawa, M., 2003, Drug News Perspect, 16:11-21.

In addition to the above recited list of growth factors, the three dimensional tissue can be characterized by the expression of Wnt proteins. Wnt is a signaling molecule having a myriad of roles in cellular pathways and cell-cell interaction processes. For example, Wnt signalling has been implicated in tumorigenesis, early mesodermal patterning of the embryo, morphogenesis of the brain and kidneys, regulation of mammary gland proliferation and Alzheimer's disease.

“Wnt” or “Wnt protein” as used herein refers to a protein with one or more of the following functional activities: (1) binding to Wnt receptors, also referred to a Frizzled proteins, (2) wnt-mediated signaling; (3) modulating phosphorylation of Dishevelled protein and cellular localization of Axin (4) modulation of cellular β-catenin levels and corresponding signaling pathway, (5) modulation of TCF/LEF transcription factors, and (6) increasing intracellular calcium and activation of Ca⁺² sensitive proteins (e.g., calmodulin dependent kinase). “Modulation” as used in the context of Wnt proteins refers to an increase or decrease in cellular levels, changes in intracellular distribution, and/or changes in functional (e.g., enzymatic) activity of the molecule modulated by Wnt.

“Wnt mediated signaling” refers to activation of a signaling activity initiated by or dependent on interaction of Wnt protein and its cognate receptor protein. As a point of reference, the canonical Wnt signaling pathway involves binding of the Wnt protein to its corresponding cellular receptor, the Frizzled proteins. Receptor activation transduces a signal by phosphorylation of the protein Dishevelled, which interacts with Axin. This interaction disrupts the formation of a cellular complex comprised of the proteins Axin, Adenomatous Polyposis Coli (APC), and glycogen synthase kinase-3β (GSK-3) that are believed to regulate β-catenin activity by promoting its degradation via a proteosome mediated pathway. Wnt signaling, through its action on Dishevelled and Axin, inhibits degradation of β-catenin, thereby leading to β-catenin accumulation in the cytoplasm and nucleus. β-catenin interacts with the transcription factor TCF/LEF and promotes its translocation into the nucleus, where the protein complex modulates the transcription of various genes.

It is to be understood, however, that Wnt signaling is not restricted to the canonical pathway, and that cells may have alternative pathways affected by signal transduction mediated by Wnt. β-catenin has beeen shown to interact with other types of transcription factors, such as p300/CBP, BRG-1, and LIM domain protein FHL-2. In addition, several non-canonical Wnt signaling pathways have been elucidated that act independently of β-catenin (see, e.g., Lustig and Behrens, 2003, J. Cancer Res. Clin. Oncol. 129:199-221; Polakis, P., 2000, Genes Dev. 14:1837-1851). In one noncannonical pathway, Wnt binds to the Frizzled receptor resulting in the activation of heterotrimeric G-proteins and subsequent mobilization of phospholipase C and phosphodiesterase. This results in a decrease in cgMP, an increase in intracellular Ca⁺², and activation of protein kinase C and other Ca⁺² regulated proteins. A second non-canonical pathway is the planar cell polarity (PCP) pathway that defines polarity in select epithelial tissues, particularly along an axis perpendicular to the apical-basal border. In vertebrates, it may contribute to the differentiation and orientation of inner ear hair cell stereocilia, and direct the expansion of mesoderm and neuroectoderm during gastrulation (Dabdoub and Kelley, 2005, J. Neurobiol. 64(4):446-57). It is thought that activation of the PCP pathway occurs by Wnt binding to Frizzled, which activates Dishevelled. Dishevelled then recruits RhoA/Rac, which ultimately leads to JNK (c-jun NH2-terminal kinase) pathway activation. A major target of the JNK pathway appears to be the AP-1 (activator protein-1) transcription factor.

“Wnt” or “Wnt proteins” are also characterized structurally by their sequence similarity or identity to mouse Wnt-1 and Wingless in Drosophila. As used herein “percentage of sequence identity” and “percentage homology” are used interchangeably to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols in Molecular Biology, John Wiley & Sons, Inc., 1995 Supplement). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

While all of the above mentioned algorithms and programs are suitable for a determination of sequence alignment and % sequence identity, in some embodiments, the BESTFIT or GAP programs in the GCG Wisconsin Software package (Accelrys, Madison Wis.),using default parameters provided, is used to determine % sequence identity.

Of relevance to the present disclosure are Wnt proteins expressed in mammals, such as rodents, felines, canines, ungulates, and primates. For instance, human Wnt proteins that have been identified share 27% to 83% amino-acid sequence identity. Additional structural characteristics of Wnt protein are a conserved pattern of about 23 or 24 cysteine residues, a hydrophobic signal sequence, and a conserved asparagine linked oligosaccharide modification sequence. Some Wnt proteins are also lipid modified, such as with a palmitoyl group (Wilkert et al., 2003, Nature 423(6938):448-52). Exemplary Wnt proteins and corresponding genes expressed in mammals include, among others, Wnt 1, Wnt 2, Wnt 2B, Wnt 3, Wnt3A, Wnt4, Wnt 4B, Wnt5A, Wnt 5B, Wnt 6, Wnt 7A, Wnt 7B, Wnt8A, Wnt8B, Wnt9A, Wnt9B, Wnt10A, Wnt11, and Wnt 16. Other identified forms of Wnt, such as Wnt12, Wnt13, Wnt14, and Wnt 15, appear to fall within the proteins described for Wnt 1-11 and 16. Protein and amino acid sequences of each of the mammalian Wnt proteins are available in databases such as SwissPro and Genbank (NCBI) (see, e.g., U.S. Publication 2004/0248803, the disclosure of which is incorporated herein by reference in its entirety).

In the embodiments herein, the repertoire of Wnt factors elaborated by the three dimensional tissues can be used to promote tissue repair. Wnt factors produced by the three dimensional tissues comprise at least Wnt5a, Wnt7a, and Wnt11. As used herein, Wnt5a refers to a Wnt protein with the functional activities described above and sequence similarity to human Wnt protein with the amino acid sequence in NCBI Accession Nos. AAH74783 (gI:50959709) or AAA16842 (gI:348918) (see also, Danielson et al., 1995, J. Biol. Chem. 270(52):31225-34). Wnt7a refers to a Wnt protein with the functional properties of the Wnt proteins described above and sequence similarity to human Wnt protein with the amino acid sequence in NCBI Accession Nos. BAA82509 (gI:5509901); AAC51319.1 (GI:2105100); and O00755 (gI:2501663) (see also, Ikegawa et al., 1996, Cytogenet Cell Genet. 74(1-2):149-52; Bui et al., 1997, Gene 189(1):25-9). Wnt11 refers to a Wnt protein with the functional activities described above and sequence similarity to human Wnt protein with the amino acid sequence in NCBI Accession Nos. BAB72099 (gI:17026012); CAA74159 (gI:3850708); and CAA73223.1 (gI:3850706) (see also, Kirikoshi et al., 2001, Int. J. Mol. Med. 8(6):651-6); Lako et al., 1998, Gene 219(1-2):101-10). As used herein in the context the specific Wnt proteins, “sequence similarity” refers to an amino acid sequence identity of at least about 80% or more, at least about 90% or more, at least about 95% or more, or at least about 98% or more when compared to the reference sequence. For instance, human Wnt7a displays about 97% amino acid sequence identity to murine Wnt7a while the amino acid sequence of human Wnt7a displays about 64% amino acid identity to human Wnt5a (Bui et al., supra).

In other embodiments, isolated and purified or partially purified Wnt proteins are used alone to promote tissue repair and/or as supplement to the conditioned medium produced from the three dimensional tissues. The Wnt proteins can be used independently, or in combination with other bioactive agents, such as growth factors, secreted by the three-dimensional tissue. The other bioactive agents can be isolated and purified or partially purified using methods known to those of skill in the art.

Within the scope of “Wnt” and “Wnt proteins” are protein fragments, variants, chimeric proteins (e.g., one or more Wnt protein domains or portions thereof fused or bonded to a heterologous polypeptide), and mutants of the identified Wnt proteins, where the fragments, variants, and mutants have the functional activities characteristic of the family of Wnt proteins.

In some embodiments, one or more of the repertoire of Wnt proteins elaborated by the three dimensional tissues are isolated by various techniques available to the skilled artisan. In some embodiments, antibodies to identified Wnt proteins may be used en mass to isolate the group of Wnt proteins produced by the three dimensional tissue. In other embodiments, an antibody to a common epitope expressed in different Wnt proteins may be used to isolated multiple Wnt proteins. In still other embodiments, antibodies to specific Wnt proteins (e.g., Wnt5a, Wnt7a, and Wnt11) can be used to isolate a single type of Wnt protein produced by the cultures. Antibodies can be immobilized in a column or to a solid surface (e.g., magnetic beads, agarose beads, etc.) to isolate the Wnt proteins or alternatively precipitated by agents such as Staph A protein or other antibody binding agents. Procedures for antibody based purification are described in many reference works, such as Ausubel, Current Methods in Molecular Biolgy, John Wiley & Sons, updates to 2005; Antibodies: A Laboratory Manual; Scopes, 1984, Protein Purification: Principles and Practice, Springer Verlag New York, Inc., N.Y.; and Livingstone, 1974, Methods In Enzymology: Immunoaffinity Chromatography of Proteins 34:723 731. All publications are incorporated herein by reference in their entireties.

In some embodiments, Wnt proteins are isolated using the procedures described in U.S. Patent Publication No. 2004/0248803, the disclosure of which is incorporated herein by reference in its entirety.

Other art known techniques for purifying proteins may also be used, such as reverse phase chromatography, high performance liquid chromatography, ion exchange chromatography, gel electrophoresis, affinity chromatography. The actual conditions used to isolate the Wnt proteins will depend, in part, on factors such as net charge, hydrophobicity, hydrophilicity, molecular weight, etc., and will be apparent to those having skill in the art.

It is to be understood that additional bioactive agents, including other growth factors, can be produced by the three dimensional tissues such that the scope of the conditioned medium produced therefrom, is not to be limited by the description above.

Genetically Engineered Cells

Genetically engineered three-dimensional tissues can be prepared as described in U.S. Pat. Nos. 5,785,964 and 5,957,972, which are incorporated herein by reference in their entireties. A genetically-engineered tissue can serve as a gene delivery vehicle for sustained release of bioactive agents, such as growth factors (soluble and matrix bound), cytokines, and/or Wnt proteins in vivo. For example, in certain embodiments, cells, such as stromal cells, can be engineered to express a gene product that is either exogenous or endogenous to the engineered cell. Stromal cells that can usefully be genetically engineered include, but are not limited to, fibroblasts (of fetal, neonatal, or adult origin), smooth muscle cells, cardiac muscle cells, stem or progenitor cells, and other cells found in loose connective tissue such as endothelial cells, macrophages, monocytes, adipocytes, pericytes, and reticular cells found in bone marrow. In various embodiments, stem or progenitor cells can be engineered to express an exogenous or endogenous gene product, and cultured on a three-dimensional scaffold, alone or in combination with stromal cells.

The cells and tissues can be engineered to express a desired gene product which can impart a wide variety of functions, including, but not limited to, enhanced function of the genetically engineered cells and tissues to promote tissue repair when implanted in vivo. The engineered gene product can be a peptide or protein, such as an enzyme, hormone, cytokine, a regulatory protein, such as a transcription factor or DNA binding protein, a structural protein, such as a cell surface protein, or the desired gene product can be a nucleic acid such as a ribosome or antisense molecule. In some embodiments, the desired gene product is one or more Wnt proteins, which play a role in differentiation and proliferation of a variety of cells as described herein (see e.g., U.S. Patent Publication Nos. 2004/0248803 and 2005/0043260, and Miller, J. R., 2001, Genome Biology 3:3001.1-3001.15; the disclosures of which are incorporated herein by reference in their entireties).

In some embodiments, engineered gene products that can be used to provide enhanced properties to the genetically engineered cells, include but are not limited to, gene products which enhance cell growth, e.g., vascular endothelial growth factor (VEGF), hepatocyte growth factor (HGF), fibroblast growth factors (FGF), platelet derived growth factor (PDGF), epidermal growth factor (EGF), transforming growth factor (TGF), connective tissue growth factor (CTGF) and Wnt factors. In embodiments in which the recombinantly engineered cells are made to express Wnt factors, specific Wnt factors for expression in the cell include, at least one of Wnt5a, Wnt7a, and Wnt11. In other embodiments, the cells and tissues are genetically engineered to express desired gene products that faciliate cell immortalization, e.g., oncogenes or telomerese.

In other embodiments, the cells and tissues are genetically engineered to express gene products which can provide protective functions in vitro such as cyropreservation and anti-desiccation properties, e.g., trehalose (U.S. Pat. Nos. 4,891,319; 5,290,765; 5,693,788). The cells and tissues can also be engineered to express gene products which provide a protective function in vivo, such as those which would protect the cells from an inflammatory response and protect against rejection by the host's immune system, such as HLA epitopes, MHC alleles, immunoglobulin and receptor epitopes, epitopes of cellular adhesion molecules, cytokines and chemokines.

The desired gene products can be engineered to be expressed by the cells and tissues described herein. The desired gene products can be engineered to be expressed constitutively or in a tissue-specific or stimuli-specific manner. The nucleotide sequences encoding the desired gene products can be operably linked, e.g., to promoter elements which are constitutively active, tissue-specific, or induced upon presence of one or more specific stimulus.

In some embodiments, the nucleotide sequences encoding the engineered gene products are operably linked to regulatory promoter elements that are responsive to shear or radial stress. In these embodiments, the promoter element is activated by passing blood flow (shear), as well as by the radial stress that is induced as a result of the pulsatile flow of blood through the heart or vessel.

Examples of other regulatory promoter elements useful in the three dimensional tissue compositions and methods described herein, include tetracycline responsive elements, nicotine responsive elements, insulin responsive elements, glucose responsive elements, interferon responsive elements, glucocorticoid responsive elements, estrogen/progesterone responsive elements, retinoid acid responsive elements, viral transactivators, early or late promoters of SV40 adenovirus, the lac system, the trp system, the TAC system, the TRC system, the promoter for 3-phosphoglycerate and the promoters of acid phosphatase. In addition, artificial response elements can be constructed, comprising multimers of transcription factor binding sites and hormone-response elements, typically similar to the molecular architecture of naturally-occurring promoters and enhancers (see, e.g., Herr and Clarke, 1986, J Cell 45(3): 461-70). Such artificial composite regulatory regions can be designed to respond to any desired signal and be expressed in particular cell-types depending on the promoter/enhancer binding sites selected.

Processing of Conditioned Media and Pharmaceutical Compositions

The conditioned medium produced by the three dimensional tissues can be used directly or further processed in various ways. The medium can be subject to lyophilization for preservation and/or concentration of the bioactive agents that promote tissue repair. A typical lyophilization process comprises three separate and interdependent processes; freezing, primary drying (sublimation), and secondary drying (desorption). Various biocompatible preservatives, cryoprotectives, and stabilizer agents can be used to preserve activity where required. Non-limiting examples of biocompatible agents include, among others, glycerol, dimethyl sulfoxide, and trehalose. The lyophilazate, in some embodiments, also includes one or more excipients such as buffers, bulking agents, and tonicity modifiers. The freeze-dried media is reconstituted by addition of a suitable solution or pharmaceutical diluent, as further described below.

In some embodiments, the conditioned medium can be processed by precipitating the bioactive agents (e.g., growth factors, cytokines, and/or Wnt proteins) in the medium. Precipitation may be performed using various procedures, such as salting out with ammonium sulfate or use of hydrophilic polymers, for example polyethylene glycol.

In other embodiments, the conditioned medium is subject to filtration using various selective filters. Processing the conditioned medium by filtering is useful in concentrating the factors that promote tissue repair and for removing small molecules and solutes used in the cconditioned medium. Filters with selectivity for specified molecular weights include <5000 daltons, <10,000 daltons, and <15,000 daltons. Other filters can be used and the processed media assayed for tissue repair promoting activities as described herein. Exemplary filters and concentrator system include those based on, among others, hollow fiber filters, filter disks, and filter probes (see, e.g., Amicon Stirred Ultrafiltration Cells, )

In still other embodiments, the conditioned medium is subject to chromatography to remove salts, impurities, or fractionate various components of the medium. Various chromatographic techniques may be employed, such as molecular sieving, ion exchange, reverse phase, affinity chromatographic techniques. For processing conditioned medium without significant loss of bioactivity, mild chromatographic media is used. Non-limiting examples include, among others, dextran, agarose, polyacrylamide based separation media (e.g., available under various tradenames, such as Sephadex, Sepharose, and Sephacryl).

In some embodiments, impurities are removed from the conditioned medium using the methods disclosed in U.S. Patent Publication No. 2004/0248803, the disclosure of which is incorporated herein by reference in its entirety.

The conditioned medium can be used directly without the addition of pharmaceutically acceptable excipients, vehicles or carriers, or pharmaceutical compositions can be prepared that comprise the conditioned medium and various pharmaceutically acceptable excipients, vehicles or carriers. A pharmaceutical composition refers to a form of the conditioned media and at least one pharmaceutically acceptable vehicle, carrier, or excipient. For intradermal, subcutaneous or intramuscular administration, the compositions may be prepared in sterile suspension, solutions or emulsions of the conditioned media in aqueous or oily vehicles. The compositions can also contain formulating agents, such as suspending, stabilizing or dispersing agents. Formulations for injection can be presented in unit dosage form, ampules in multidose containers, with or without preservatives. Alternatively, the compositions can be presented in powder form for reconstitution with a suitable vehicle including, by way of example and not limitation, sterile pyrogen free water, saline, buffer, or dextrose solution.

In still other embodiments, the conditioned medium comprising the bioactive agents can be introduced or encapsulated into the lumen of liposomes for delivery and for extending the life time of the bioactive agents. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which can be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids ; and other lipids (see, e.g., U.S. Pat. No. 5,833,948).

Cationic lipids are also suitable for forming liposomes. Generally, the cationic lipids have a net positive charge and have a lipophilic portion, such as a sterol or an acyl or diacyl side chain. In some embodiments, the head group is positively charged. Typical cationic lipids include 1,2-dioleyloxy-3-(trimethylamino) propane; N-[1-(2,3,-ditetradecycloxy)propyl]-N,N-dimethyl-N-N-hydroxyethylammonium bromide; N-[1-(2,3-dioleyloxy)propyl]-N,N-dimethyl-N-hydroxyethylammonium bromide; N-[1-(2,3-dioleyloxy)propyl]-N,N,N-trimethylammonium chloride; 3-[N-(N′,N′-dimethylaminoethane)carbamoyl]choiesterol; and dimethyldioctadecylammonium.

In other embodiments, the liposomes are fusogenic liposomes, which are characterized by their ability to fuse with a cell membrane upon appropriate change in physiological condition or by presence of fusogenic component, particularly a fusogenic peptide or protein. In some embodiments, the fusogenic liposomes are pH and/or temperature sensitive in that fusion with a cell membrane is affected by change in temperature and/or pH (see, e.g., U.S. Pat. Nos. 4,789,633 and 4,873,089, 6,200,598 and 6,726,925). Generally, pH sensitive liposomes are acid sensitive. Thus, fusion is enhanced in physiological environments where the pH is mildly acidic, for example the environment of a lysosome, endosome and inflammatory tissues. This property allows direct release of the liposome contents into the intracellular environment following endocytosis of liposomes (Mizoue, T., 2002, Int. J. Pharm. 237: 129-137).

Liposomes also include vesicles derivatized with a hydrophilic polymer, as provided in U.S. Pat. No. 5,013,556 and 5,395,619, hereby incorporated by reference, (see also, Kono, K. et al., 2000, J. Controlled Release 68: 225-35; Zalipsky et al., 1995, Bioconjug. Chem. 6: 705-708) to extend the circulation lifetime in vivo. Hydrophilic polymers for coating or derivation of the liposomes include polyethylene glycol, polyvinylpyrrolidone, polyvinylmethyl ether, and polyaspartamide. Other types of suitable coatings will be apparent to the skilled artisan.

Liposomes are prepared by methods well known in the art (see for example, Szoka et al., 1980, Ann. Rev. Biophys. Bioeng. 9: 467-508). One typical method is the lipid film hydration technique in which lipid components are mixed in an organic solvent followed by evaporation of the solvent to generate a lipid film. Hydration of the film in aqueous buffer solution, preferably containing the subject polypeptide or nucleic acid, results in an emulsion, which is sonicated or extruded to reduce the size and polydispersity. Other methods include reverse-phase evaporation (see, e.g., Pidgeon et al., 1987, Biochemistry 26:17-29; Duzgunes et al., 1983, Biochim. Biophys. Acta. 732:289-99), freezing and thawing of phospholipid mixtures, and ether infusion.

For topical administration, the bioactive agent(s) can be formulated as solutions, gels, ointments, creams, and suspensions, as are well-known in the art. In some embodiments, the conditioned media can be applied via transdermal delivery systems, which slowly release the bioactive agent(s) for percutaneous absorption. Permeation enhancers may be used to facilitate transdermal penetration of the active factors in the conditioned media. Transdermal patches are described in for example, U.S. Pat. Nos. 5,407,713.; 5,352,456; 5,332,213; 5,336,168; 5,290,561; 5,254,346; 5,164,189; 5,163,899; 5,088,977; 5,087,240; 5,008,110; and 4,921,475.

Additionally, one or more binding protein/peptide(s) can be partially entrapped in the particulate polymeric matrix upon formation thereof. Under these circumstances, such entrapped binding protein/peptide(s) provides residual selective binding character to the particulate. Mild particulate formation conditions, such as those employed by Cohen et al., 1991, Pharmaceutical Research 8:713-720 can be used to retain the activity of the factors in the conditioned media. Such entrapped binding protein is also useful in target cell reattachment of a partially degraded particulate that has undergone exocytosis. Other polymeric particulate dosage forms (e.g., non-biodegradable dosage forms) having different exposed functional groups can be bound to binding proteins or peptides in accordance with the principles discussed above.

The conditioned medium can be used alone, or in combination with other compatible bioactive agents useful for promoting tissue repair. In some embodiments, as further described below, the conditioned medium can be used adjunctively with other compounds or compositions used, for example, to treat cardiovascular disease. In some embodiments, the adjunctive agent comprises a modulator of VEGF activity.

Use of Bioactive Agents Produced From Cultured Three Dimensional Tissues to Promote Tissue Repair

The conditioned medium, or components thereof, such as the repertoire of Wnt factors elaborated by the three dimensional tissues can be used to promote and/or repair damaged tissue. Tissue damage and defects can result from many conditions, including, but not limited to, disease, surgery, environmental exposure, injury, and aging. For example, tissue damage can result from ischemia, typically caused by a shortage of blood and oxygen to a muscle. Generally, by “tissue repair promoting activities and/or characteristics” herein is meant that the application of the conditioned medium, or components thereof facilitates one or more of the following physiological changes within the damaged tissue: (1) angiogenesis and/or revascularization; (2) stimulation and/or growth of new tissue; (3) endothelialization; and (4) improvement in the number of tight junctions; (5) increase in cellular processes and microvilli; and, (6) partial or complete restoration of one or more functions of the treated tissue to levels observed in the corresponding healthy tissue. For example, if the treated tissue is cardiac tissue, an improvement in the pumping efficiency of the heart as measured by the ejection fraction or cardiac output can be observed. Revascularization can be measured by noninvasive means such as ultrasound imaging routinely used by those skilled in the art.

In some embodiments, the damaged tissue includes cardiac muscle tissue, skeletal muscle tissue, brain tissue (e.g., affected by stroke or malformations of the arteries and veins covering the brain (i.e., AV malformations)), kidney, liver, organs of the gastrointestinal tract, muscle tissue afflicted by atrophy, including neurologically based muscle atrophy and lung tissue. In further embodiments, the damaged tissue is present in a mammal, such as a human.

In other embodiments, the damaged tissue includes but is not limited to skin, including tissue wounds, such as skin ulcers and burns, and hair follicles that are no longer undergoing hair shaft production.

In some embodiments, the damaged tissue can be artificially created, i.e., can be created as a result of a surgical procedure.

In various embodiments, the repertoire of proteins produced by the three dimensional tissues, and typically present additionally in the conditioned medium, comprises at least one of Wnt5a, Wnt7a, and Wnt11, typically each of Wnt 5a, Wnt7a and Wnt 11 (described above).

In some embodiments, the conditioned medium, or components thereof, such as isolated Wnt proteins, find uses in promoting the repair of damaged heart tissue. In some embodiments, the conditioned medium, or components thereof can be administered to the damaged heart tissue. In other embodiments, the conditioned medium can be administered in combination with other therapies, see, e.g., U.S. patent application Ser. No. ______, entitled “Methods for Treating Ischemic Tissue,” filed concurrently herewith and incorporated herein by reference in its entirety.

In some embodiments, the conditioned medium, or components thereof, can be administered to damaged heart tissue in combination with a cultured three-dimensional tissue, the latter comprising stem cells (see, e.g., U.S. patent application Ser. No. ______, entitled “Methods for Treating Ischemic Tissue,” filed concurrently herewith and incorporated herein by reference in its entirety). In some embodiments, the damaged tissue is heart tissue. While not intending to be bound by theory, it is believed that this method facilitates the recruitment and/or differentiation of stem or progenitor cells into functional cardiac tissue. Suitable methods for measuring the improvement of damaged heart tissue following the administration of the conditioned medium are described in U.S. patent application Ser. No. ______, entitled “Methods for Treating Ischemic Tissue,” filed concurrently herewith and incorporated herein by reference in its entirety.

In some embodiments, the conditioned medium, or components thereof, such as isolated Wnt proteins, find uses in promoting the repair of damaged intestinal tissue, such as tissue present in the small intestine, the large intestine, the colon, or combinations thereof. In some embodiments, the damaged intestinal tissue is present in individuals diagnosed with a variety of bowel disorders, such as Crohn's disease, irritable bowel disorder, and ulcerative colitis. Following treatment, improvements in the functioning of damaged intestinal tissue can be measured by changes in the permeability of the intestine due to an increase in intercellular adherens and tight junctions. Additional tests for measuring changes intestinal permeability are discussed in Bjarnason, 1994, Gut, 35 (1Suppl):S18-22, and DeMeo et al., 2002, J. Clin Gastroenterol, 34(4):385-96, Korzenik, J R, 2005, J Clin Gastroenterol, 39(4 Suppl 2):S59-65, the disclosures of which are incorporated herein by reference in their entireties. Other methods that can be used to measure improvements in the functioning of damaged intestinal tissue include, but are not limited to, comparing the number and/or location of lesions before and after treatment, patient reports of well-being following treatment, reduction in symptoms of diarrhea, bloating, cramping, and bleeding following treatment.

In some embodiments, the damaged intestinal tissue can be treated by contacting the damaged intestinal tissue with a cultured three-dimensional tissue as described in U.S. patent application Ser. No. ______, entitled “Methods for Treating Ischemic Tissue,” and U.S. patent application Ser. No. ______, entitled “Three Dimensional Tissues and Uses Thereof,” filed concurrently herewith and incorporated by reference in their entireties. In these embodiments, the conditioned medium, or components thereof, such as isolated Wnt proteins, can be applied to the tissue concurrently with, or following treatment with the cultured three-dimensional tissue. For example, in some embodiments, the conditioned medium can be administered at selective times following treatment with the cultured three-dimensional tissue to maintain the treated intestinal tissue in a non-diseased state. Improvements in the functioning of damaged intestinal tissue following treatment can be measured as described above.

In some embodiments, the conditioned medium, or components thereof, such as isolated Wnt proteins, find uses in enhancing hair growth in subjects in whom additional hair growth is desired or desirable. Embodiments are described, for example, in U.S. patent application Ser. No. ______ entitled, “Compositions and Methods for Promoting Hair Growth,” filed concurrently herewith and incorporated herein by reference in its entirety.

Dosage

The bioactive agent(s) typically will be used in an amount effective to treat the damaged tissue of interest. The bioactive agent(s) can be administered therapeutically to achieve therapeutic benefit or prophylactically to achieve prophylactic benefit. By therapeutic benefit is meant eradication or amelioration of the underlying condition or disorder being treated. Therapeutic benefit also includes halting or slowing the progression of the disease, regardless of whether improvement is realized.

For prophylactic administration, the active compound may be administered to a patient at risk of developing a disorder characterized by, caused by or associated with the formation of damaged tissue, such as the various disorders above-described. For example, the conditioned media can be administered prior to appearance of symptoms of damaged tissue or after the first signs of tissue damage to avoid worsening of the condition. Prophylactic administration can be performed to avoid the onset of symptoms in a patient diagnosed with an underlying disorder. Active compounds can also be administered to healthy individuals where the administration is for cosmetic purposes, for example, to treat or prophylax against hair loss.

The amount of conditioned medium, or of factors partially or completely purified or isolated therefrom, to be administered will depend upon a variety of factors, including, for example, the particular indication being treated, the mode of administration, whether the desired benefit is prophylactic or therapeutic, the severity of the indication being treated and the age and weight of the patient, and effectiveness of the dosage form. Determination of an effective dosage is well within the capabilities of those skilled in the art.

Initial dosages can be estimated initially from in vitro assays. For example, initial dosages can be formulated using the angiogenesis assays described in U.S. Publication No. 20040219134 and U.S. patent application Ser. No. ______, entitled “Methods for Treating Ischemic Tissue,” filed currently herewith and incorporated herein by reference in its entirety. Initial dosages can also be estimated from in vivo data, such as animal models. Animals models useful for testing the efficacy of compounds include rodents, primates, and other mammals, such as pigs, rabbits and dogs. Ordinarily skilled artisans can routinely adapt such information to determine dosages suitable for human administration.

Dosage amounts will depend upon, among other factors, the activity of the conditioned medium, the mode of administration, the condition being treated, and various factors discussed above. Dosage amount and interval may be adjusted individually to provide levels sufficient to maintain therapeutic or prophylactic effect. The compositions can be administered as often as necessary, e.g., one time daily, two or more times daily, once per week, two or more times per week, once a month, two or more times a month, depending upon, among other things, the indication being treated, the level of tissue repair desired, and the judgment of the prescribing physician. The compositions will provide therapeutic or prophylactic benefit without causing substantial toxicity or adverse immunological reaction. Skilled artisans will be able to optimize effective local dosages without undue experimentation.

The spatial density of administration (e.g., local administration for a defined surface area) will depend on the type of tissue, degree of tissue damage, the level of tissue repair desired, the volume of conditioned medium administered, and the efficacy of the composition. Various embodiments utilizing different spatial densities for the administration of conditioned medium, or of factors partially or completely purified or isolated therefrom, are described in U.S. patent application Ser. No. ______, entitled “Three Dimensional Tissues and Uses Thereof,” filed concurrently herewith and incorporated by reference in its entirety.

Kits

Further provided herein are kits comprising the conditioned medium, or processed fractions thereof, in various forms, as described herein. The kit can contain liquid, solid, or powder formulations of the conditioned medium, in format suitable for administration. In various embodiments, suitable diluents for reconstituting the composition can be included in the kits for preparing the composition for administration. The kits can further comprise devices for administration, such as injection devices (e.g., manually, mechanically or electronically driven), for use by the person skilled in the art. When other compounds are administered adjunctively with the conditioned medium, they can also be included in the kits. Typically accompanying the compositions are instructions for preparing and administering of the compositions. These may be in various formats, such a printed matter, magnetic disk, magnetic tape, compact disc, flash memory, or other media suitable for conveying the appropriate information.

All literature and similar materials cited in this application, including but not limited to patents, patent applications, articles, books, and treatises, regardless of the format of such literature and similar materials, are expressly incorporated by reference in their entireties for any purpose. In the event that one or more of the incorporated literature and similar materials differs from or contradicts this disclosure, including but not limited to defined terms, term usage, described techniques, or the like, this application controls.

All numerical ranges in this specification are intended to be inclusive of their upper and lower limits.

Unless defined otherwise, all technical and scientific terms used herein have the same meaning as commonly understood by one of ordinary skill in the art to which this invention belongs. Unless mentioned otherwise the techniques employed or contemplated herein are standard methodologies well known to one of ordinary skill in the art. The materials, methods and examples are illustrative only and not limiting.

### EXAMPLES

### Example 1

### Three Dimensional Tissue Conditioned Medium Promoted Differentiation and Repair of Epithelial Cells

Materials And Methods

Epithelial Cell Growth and Differentiation Assay. This study evaluated the effects of three-dimensional tissue conditioned medium on four different human epithelial cell lines in vitro. The cell lines utilized were as follows: (1) primary human epidermal keratinocytes at passage 2-3 (P2-3), (2) an immortalized intestinal epithelial cell line (Caco-2), (3) an immortalized respiratory epithelial cell line (NCI-H292), and (4) an immortalized colonic epithelial cell line (HT-29). These immortalized cell lines are accepted models of the tissue from which they were derived, and are well characterized in vitro. Epidermal keratinocytes represent a primary cell type.

Cell proliferation. Cell lines were obtained from ATCC and plated at approximately 5000 cells/cm² in 24 well plates and cultured for 2-3 days in the presence of 10% (v/v) test materials or control non-conditioned medium. Primary human epidermal keratinocytes at P2 in KGM-2 medium (Clonetics, Inc.) were also evaluated in some experiments. Controls consisted of untreated cells as well as non-conditioned medium as a negative control. After rinsing briefly in PBS, relative cell number was estimated by DNA fluorescence using Molecular Probes, Inc. CyQuant Cell Proliferation kit, and data is expressed as relative DNA fluorescence per well, with 6 wells per condition. Experiments were repeated twice for each cell line. In the studies herein, conditioned medium is referred to as Nouricel.

Immunofluorescence and Phase-contrast Microscopy. Primary human epidermal keratinocytes at passage 2 were cultured in serum free medium supplemented with 10% (v/v) control non-conditioned medium or test substances for 2 days on collagen coated glass chamber slides. Cultures were rinsed, fixed in paraformaldehyde, permeabilized in 0.1% Tween-20 in PBS. Primary antibodies were as follows: Mab x ZO-1 and Rb x Claudin-1 (Zymed, Inc.) at 2 μg/ml each for 30 minutes followed by detection with fluorochrome conjugated secondary antibodies and mounting in Vectashield (Vector Labs, Inc.) with DAPI added as a nuclear counterstain. Live cultures were photographed prior to fixation under phase contrast illumination at approximately 100× magnification.

Transmission Electron Microscopy on Organotypic Cell Cultures. Cells were cultured on collagen coated microporous membranes for 10 days after seeding at high cell density (1 00,000/cm²), conditions known to support differentiation of the Caco-2 cell line, in the presence of 10% (v/v) of the <10 kD three-dimensional stromal tissue conditioned medium Permeate or an untreated control. Both the Caco-2 and NCI-H292 cell lines were evaluated. Cultures were rinsed and then fixed in modified Karnovsky's fixative, and processed for electron microscopy using standard techniques.

Expression Analysis by Fluorescent Gene Chip Arrays. Single low-density cultures of epidermal keratinocytes, Caco-2 cells, and NCI-H292 cells in 10 cm dishes were treated with concentrated three-dimensional stromal tissue conditioned medium or a non-conditioned medium control fro 3-5 days, and mRNA was isolated. After conversion to cDNA and fluorescent labeling, pooled cDNA's were hybridized to gene-chip arrays. Fluorescence detection of hybridized probes to approximately 10,000 known gene transcripts was conducted at UC Irvine and the data was analyzed using Genespring software. Normalized fluorescence was graphed comparing the relative change in expression profiles.

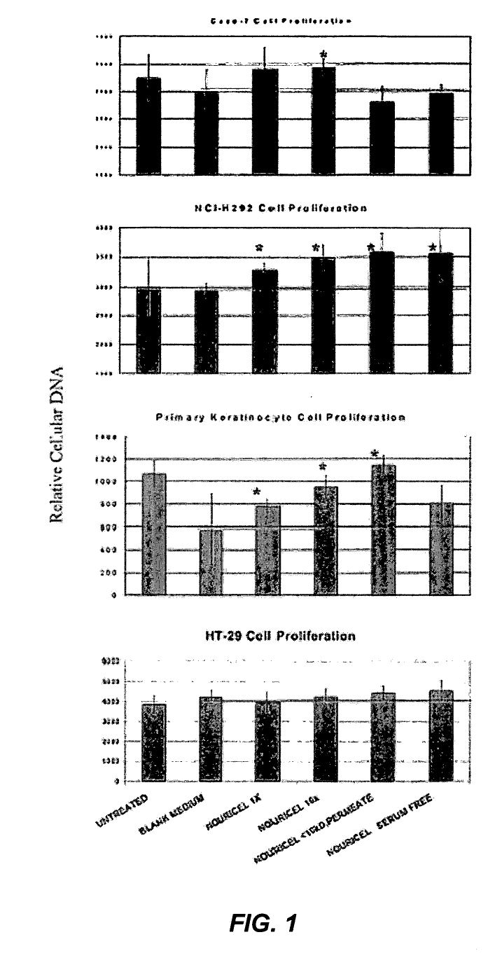

Results. A comparison of the effects of three-dimensional stromal tissue conditioned medium on cell proliferation in four different epithelial cell types is shown in FIG. 1. Not all cell lines responded and not all conditioned medium were effective. The effects of three-dimensional stromal tissue conditioned medium on cell proliferation were cell type-specific in that three-dimensional stromal tissue conditioned medium enhanced cell growth in both primary epidermal keratinocytes and NCI-H292 cells, but not for the most part in Caco-2 or HT-29 cells. When these three-dimensional stromal tissue conditioned medium treated cultures were examined by phase contrast microscopy, again the effects were cell type-specific in that HT-29 cells were not altered morphologically, but keratinocytes (data not shown), Caco-2, and NCI-H292 cells were altered. Both the Caco-2 and NCI-H292 cell lines displayed the formation of dome-like structures when examined by phase contrast microscopy. The Caco-2 cell line also displayed altered localization of both adeherens junction (ZO-1) and tight junction markers by immunofluorescence after three-dimensional stromal tissue conditioned medium treatment.

Since the formation of intercellular adherens and tight junctions is a key component of epithelial cell differentiation, cells were evaluated three-dimensional stromal tissue conditioned medium's effects on Caco-2 and NCI-H292 cell lines in high-density organotypic cultures using microporous inserts to promote apical/basolateral differentiation and junction formation. Analysis of these cultures by transmission electron microscopy (TEM) revealed a number of apparent effects of the >10 kD permeate from the three-dimensional stromal tissue conditioned medium concentration process on cellular differentiation.

The apparent formation of dome-like structures in the Caco-2 and NCI-H292 cell lines may indicate an enhancement or induction of differentiation, as mucin-production (a marker of both intestinal and respiratory epithelium) has been reported to lead to similar morphological changes in these cell lines as well as primary human intestinal and respiratory cells in culture.

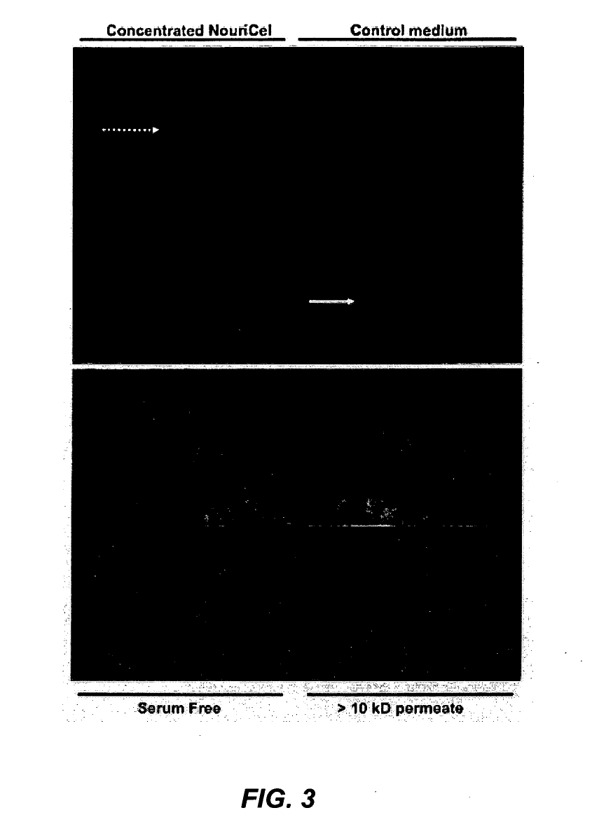

Since the formation of these putative differentiated structures is accompanied by alterations in intercellular junctions, we evaluated the effects of conditioned medium on adherens and tight junction markers in Caco-2 cells by immunofluorescence. The results of this experiment are shown in FIG. 3, which depicts immunofluorescence analysis for adherens (ZO-1) and tight (claudin-1) junction markers in Caco-2 cells treated with three-dimensional stromal tissue conditioned medium on collagen coated glass slides. Note the discontinuous staining for ZO-1 in the control medium panel (white arrow), and the junctional localization of claudin-1 in all the three-dimensional stromal tissue conditioned medium treated panels (dashed white arrow).

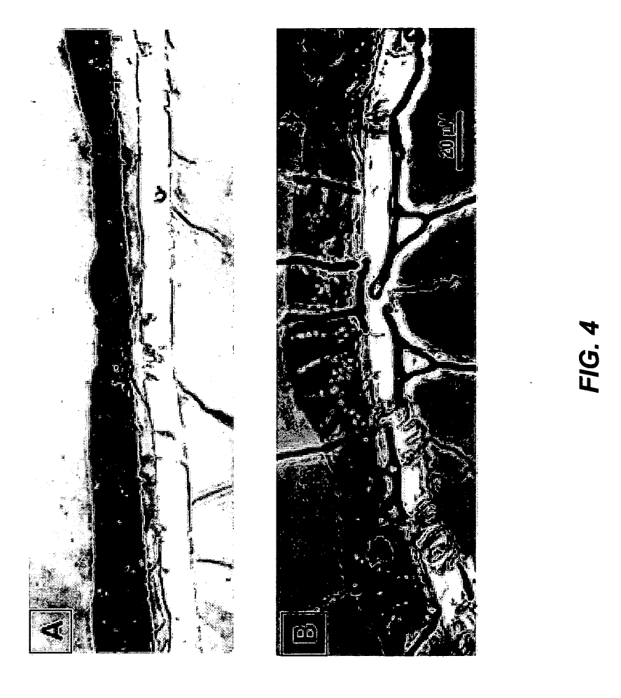

Since simple epithelial differentiation displays number of well-characterized morphological changes that can best be identified by transmission electron microscopy (TEM), and since standard monolayer cultures are not favorable for this differentiation pathway, TEM analysis was performed on three-dimensional stromal tissue conditioned medium treated organotypic, high-density microporous membrane cultures. A thick-section at is shown for control and three-dimensional stromal tissue conditioned medium treated Caco-2 cells (FIG. 4). There is a difference in overall thickness, increased columnar shape, and increased intercellular spaces in cells treated with three-dimensional stromal tissue conditioned medium. These are all characteristics of normal differentiation of these cell types.

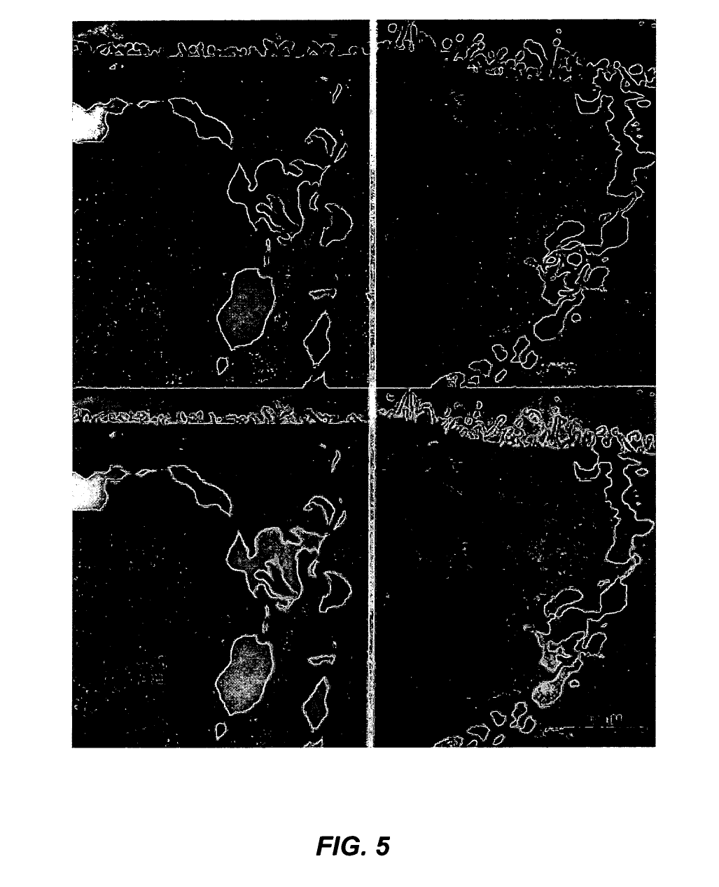

Under much higher magnification, a number of features altered after three-dimensional stromal tissue conditioned medium treatment are observed. FIG. 5, which shows TEM analysis of the effects of conditioned medium <10 kD permeate on Caco-2 cells in high density organotypic cultures. The lower duplicate panels have highlighted the following features: nuclear membranes, brushborder microvilli, mitochondria, and cellular processes. Note the increase in cellular processes, microvilli, and mitochondrial location (apical in the three-dimensional stromal tissue conditioned medium sample). Although not highlighted, tight junctions were less frequent in the conditioned medium sample than the control.

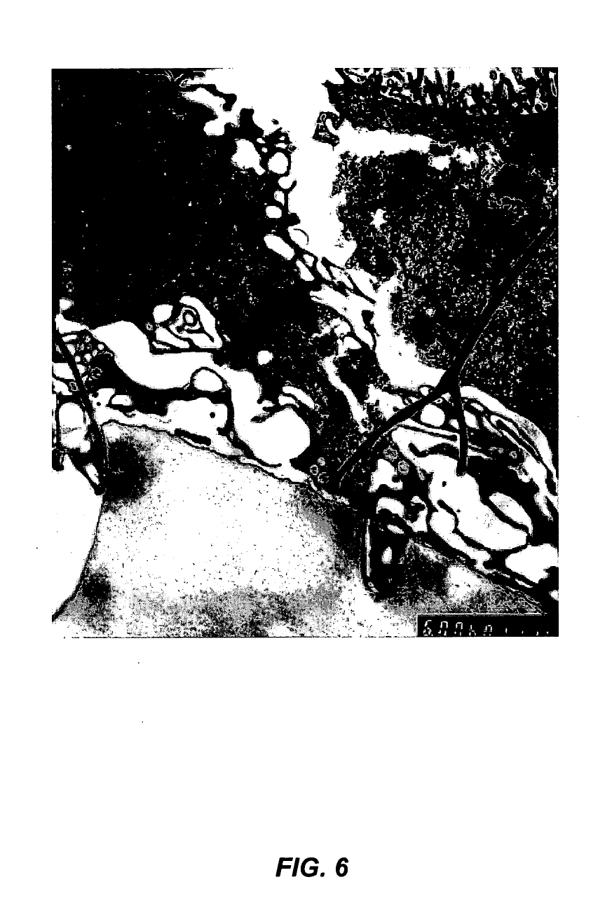

FIG. 6 is a TEM analysis of effect of conditioned medium <10 kD permeate on Caco-2 cells in high density organotypic cultures showing effects on cellular processes, apical microvilli, and dense glycogen deposits. Micrographs show the high degree of cellular processes apparently induced by three-dimensional stromal tissue conditioned medium in these cultures of Caco-2 cells. Glycogen deposits and dense microvilli can also be seen.

In a preliminary screen, the effects of concentrated three-dimensional stromal tissue conditioned medium on global gene expression were examined for cultures of epidermal keratinocytes, Caco-2 cells, and NCI-H292 cells (as a supplement). When compared to a non-conditioned medium control, three-dimensional stromal tissue conditioned medium altered the expression level of genes in three cell types (epidermal keratinocytes, Caco-2 cells, and NCI-H292 cells), although in the relative rank order of keratinocytes>NCI-H292>>Caco-2 cells.

The results above suggest that the conditioned medium contains activity (or activities) that can affect some epithelial cells in vitro. Three-dimensional stromal tissue conditioned medium can enhance cellular proliferation of epithelial cells in a cell line-specific manner, although not all versions of conditioned medium are effective. Three-dimensional stromal tissue conditioned medium can also alter epithelial cell morphology in both low-density monolayer and organotypic cell cultures at both the light and electron microscopic levels. Some of these changes are consistent with enhanced differentiation.

### Example 2

### Injection of Stromal Tissue Conditioned Medium in a Mouse Hair Model

This study evaluated the effects of conditioned medium from three dimensional tissue comprised of fibroblasts on hair follicle development in C57B1/6 mice. The experiments sought to examine whether the medium contained factors mimicking inductive signals from dermal papilla cells to induce hair growth. Candidates for hair growth activity induced by dermal fibroblast conditioned medium include Wnt gene products because (1) Wnt-signaling occurs through nuclear β-catenin during fetal development; (2) stabilized β-catenin mutant transgenes form pilosebaceous tumors in mice and humans, and (3) transplanted dermal papilla can induce follicles in rodents and this requires Wnt-signals. Also, anagen induction is thought to involve KGF/FGF-7, hormones (T3, PTH, androgens), sonic hedgehog, and Wnt gene products.

C57B1/6 mice are suitable for hair studies as they exhibit synchronized hair follicle cycling, an extended telogen phase from about days P45 to P65, and exhibit follicular melanocytes in bulb only during anagen such that the start of anagen is visually evidenced by a dark skin color. The study examined the effects of conditioned medium on hair growth by single injection subcutaneously (SQ) in the dorsal skin of approximately 7 week female C57B1/6 mice (telogen at dorsal injection site). Histology and photography were performed at days 14 (all groups) and 30 (control, neat) N=3. Test groups included Blank Medium Control, 10× neat, 10× diluted 1/10, 1/100 permeate form 10 kD concentration, topically applied, and experimental serum-free conditioned medium. The tissues were examined for formation of hair follicles.

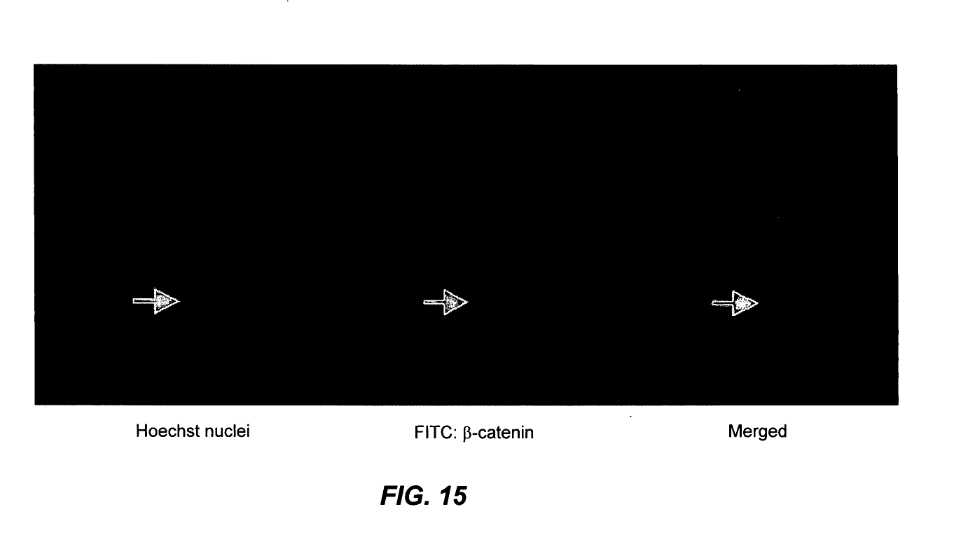

In vitro analyses of Wnt signaling in epidermal keratinocytes in vitro were also performed. FIG. 15 shows Wnt signaling in epidermal keratinocytes in vitro. Nuclear translocation of β-catenin is induced by conditioned medium, providing strong evidence that the conditioned medium contains Wnt proteins.

The results in mice show that conditioned medium made from three dimensional tissues is active inducing hair growth when injected into the skin. Hair follicle development is localized to the injection site. Wnt and Wnt-mediated signaling activity is a possible mechanism behind the hair growth in mice treated with the conditioned media because (a) Wnt genes are expressed by fibroblasts in gene chips, and (b) conditioned medium induces Wnt signaling in epidermal keratinocytes in vitro.

1. A method of treating a damaged tissue comprising, contacting the damaged tissue with a composition that comprises medium from the culturing of a three-dimensional tissue under conditions suitable for the secretion of one or more bioactive agents by the three-dimensional tissue.

2. A method of treating a damaged tissue comprising, contacting the damaged tissue with a composition comprising at least one bioactive agent at least partially purified from medium obtained from the culturing of a three-dimensional tissue under conditions suitable for the secretion of one or more bioactive agents by the three-dimensional tissue.

3. The method of claim 1 in which the composition comprises at least one Wnt protein.

4. The method of claim 3 in which one or more Wnt proteins is selected from the group consisting of Wnt5a, Wnt7a, and Wnt11a.

5. The method of claim 3 in which the composition further comprise one or more growth factors.

6. The method of claim 5 in which the growth factors are selected from the group consisting of vascular endothelial growth factor (VEGF), hepatocyte growth factor (HGF), fibroblast growth factors (FGF), platelet derived growth factor (PDGF), epidermal growth factor (EGF), connective tissue growth factor (CTGF), and transforming growth factor (TGF).

7. The method of claim 1 in which the damaged tissue is heart tissue.

8. The method of claim 1 in which the damaged tissue is ischemic tissue.

9. The method of claim 8 in which the ischemic tissue is ischemic heart tissue.

10. The method of claim 1 in which the damaged tissue is intestinal tissue.

11. The method of claim 10 in which the damaged intestinal tissue is in the small intestine, the large intestine, the colon, or combinations thereof.

12. The method of claim 2 in which the three-dimensional tissue comprises fibroblasts.

13. The method of claim 1 in which the three-dimensional tissue comprises stem cells.

14. A method of making a composition comprising one or more bioactive agents, comprising the steps of:
a) culturing a three-dimensional tissue under conditions suitable for the secretion of the bioactive agents into the growth medium;
b) at least partially purifying one or more bioactive agents from the growth medium; and,
c) adding the one or more partially purified bioactive agents to a pharmaceutically acceptable carrier.

15. The method of claim 14 in which one or more of the bioactive agents is a Wnt protein.

16. The method of claim 15 in which one or more Wnt proteins are selected from the group consisting of Wnt5a, Wnt7a, and Wnt11a.

17. The method of claim 15 in which the composition further comprise one or more growth factors.

18. The method of claim 17 in which the growth factors are selected from the group consisting of vascular endothelial growth factor (VEGF), hepatocyte growth factor (HGF), fibroblast growth factors (FGF), platelet derived growth factor (PDGF), epidermal growth factor (EGF), connective tissue growth factor (CTGF), and transforming growth factor (TGF).

19. The method of claim 14 in which the three-dimensional tissue comprises fibroblasts.

20. The method of claim 14 in which the three-dimensional tissue comprises stem cells.
